(19) United States
(12) Patent Application Publication
(10) Pub. No.: US 2017/0283889 A1
(43) Pub. Date: Oct. 5, 2017

(54) Biomarkers for HBV treatment response

(71) Applicant: Hoffmann-La Roche Inc., Little Falls, NJ (US)

(72) Inventors: Lore Gruenbaum, Cos Cob, CT (US); Cynthia Wat, London (GB); Vedran Pavlovic, London (GB); Hua He, London (GB)

(73) Assignee: Hoffmann-La Roche Inc., Little Falls, NJ (US)

(21) Appl. No.: 15/624,813

(22) Filed: Jun. 16, 2017

Related U.S. Application Data
(63) Continuation of application No. PCT/EP2015/079719, filed on Dec. 15, 2015.

(30) Foreign Application Priority Data
Dec. 18, 2014 (EP) 14198794.1

(51) Int. Cl. C12Q 1/70; A61K 38/21

(52) U.S. Cl. CPC C12Q 1/706; A61K 38/212; C12Q 2600/172; C12Q 2600/106

(57) ABSTRACT

The present invention relates to methods that are useful for predicting the response of hepatitis B virus (HBV) infected patients to pharmacological treatment.

### CROSS REFERENCE TO RELATED APPLICATIONS

This application is a continuation of International Patent Application No. PCT/EP2015/079719, having an international filing date of Dec. 15, 2015, the entire contents of which are incorporated herein by reference, and which claims benefit under 35 U.S.C. 119 to European Patent Application No. 14198794.1 filed Dec. 18, 2014.

### BACKGROUND OF THE INVENTION

The present invention relates to methods that are useful for predicting the response of hepatitis B virus (HBV) infected patients to pharmacological treatment.

The hepatitis B virus (HBV) infects 350-400 million people worldwide; one million deaths resulting from cirrhosis, liver failure, and hepatocellular carcinoma due to the infection are recorded annually. The infecting agent, hepatitis B virus (HBV), is a DNA virus which can be transmitted percutaneously, sexually, and perinatally. The prevalence of infection in Asia (>8%) is substantially higher than in Europe and North America (<2%) (Dienstag J. L., Hepatitis B Virus Infection., N. Engl. J. Med. 2008; 359: 1486-1500). The incidence of HBV acquired perinatally from an infected mother is much higher in Asia, leading to chronic infection in >90% of those exposed (WHO Fact Sheet No 204; revised August 2008). Additionally, 25% of adults who become chronically infected during childhood die from HBV-related liver cancer or cirrhosis (WHO Fact Sheet No 204; revised August 2008). Interferon alpha (IFNα) is a potent activator of anti-viral pathways and additionally mediates numerous immuno-regulatory functions (Muller U., Steinhoff U., Reis L. F. et al., Functional role of type I and type II interferons in antiviral defense, Science 1994; 264: 1918-21).

The efficacy of PEGASYS® (Pegylated IFN alfa 2a 40 KD, Peg-IFN) at a dose of 180 m/week in the treatment of HBV was demonstrated in two large-scale pivotal studies. One study was in HBeAg-negative patients (WV16241) and the other in HBeAg-positive patients (WV16240).

WV16241 was conducted between June 2001 and August 2003; 552 HBeAg-negative CHB patients were randomized to one of three treatment arms: PEG-IFN monotherapy, PEG-IFN plus lamivudine or lamivudine alone for 48 weeks. Virologic response (defined as HBV DNA<20,000 copies/mL) assessed 24 weeks after treatment cessation was comparable in the groups that received PEG-IFN (43% and 44%) and both arms were superior to the lamivudine group (29%) (Marcellin P., Lau G. K., Bonino F. et al., Peginterferon alfa-2a alone, lamivudine alone, and the two in combination in patients with HBeAg-negative chronic hepatitis B, N. Engl. J.Med. 2004; 351: 1206-17).

Study WV16240 was conducted between January 2002 and January 2004. In this study, 814 HBeAg-positive CHB patients were randomized to the same treatment arms as in WV16241, i.e. PEG-IFN monotherapy, PEG-IFN plus lamivudine or lamivudine alone for 48 weeks. Responses assessed 24 weeks after treatment cessation showed a 32% rate of HBeAg seroconversion in the PEG-IFN monotherapy group compared to 27% and 19% with PEG-IFN+ lamivudine and lamivudine monotherapy respectively (Lau G. K., Piratvisuth T,. Luo K. X. et al., Peginterferon Alfa-2a, Lamivudine, and the Combination for HBeAg-Positive Chronic Hepatitis B, N. Engl. J. Med. 2005; 352: 2682-95). Metaanalysis of controlled HBV clinical studies has demonstrated that PEG-IFN-containing treatment facilitated significant HBsAg clearance or seroconversion in CHB patients over a lamivudine regimen (Li W. C., Wang M. R., Kong L. B. et al., Peginterferon alpha-based therapy for chronic hepatitis B focusing on HBsAg clearance or seroconversion: a meta-analysis of controlled clinical trials, BMC Infect. Dis. 2011; 11: 165-177).

More recently, the Neptune study (WV19432) was conducted between May 2007 and April 2010 and compared PEG-IFN administered as either 90 or 180 μg/week administered over either 24 or 48 weeks in HBeAg-positive patients (Liaw Y. F., Jia J. D., Chan H. L. et al., Shorter durations and lower doses of peginterferon alfa-2a are associated with inferior hepatitis B e antigen seroconversion rates in hepatitis B virus genotypes B or C, Hepatology 2011; 54: 1591-9). Efficacy was determined at 24 weeks following the end of treatment. This study, demonstrated that both the lower dose and shorter durations of treatment were inferior to the approved dose and duration previously used in the WV16240 study, thus confirming that the approved treatment regimen of i.e. 180 m/week for 48 weeks is the most beneficial for patients with HBeAg-positive CHB.

However, despite the fact that PEG-IFN has been successfully used in the treatment of CHB, little is known of the impact of host factors (genetic and non-genetic) and viral factors on treatment response.

Although viral and environmental factors play important roles in HBV pathogenesis, genetic influence is clearly present. While small genetic studies have suggested the possible implications of host immune/inflammation factors (e.g. HLA, cytokine, inhibitory molecule) in the outcomes of HBV infection, a genome-wide association study (GWAS) clearly demonstrated that 11 single nucleotide polymorphisms (SNPs) across the human leukocyte antigen (HLA)-DP gene region are significantly associated with the development of persistent chronic hepatitis B virus carriers in the Japanese and Thai HBV cohorts (Kamatani Y., Wattanapokayakit S., Ochi H. et al., A genome-wide association study identifies variants in the HLA-DP locus associated with chronic hepatitis B in Asians. Nat. Genet. 2009; 41: 591-595). Subsequently this finding was also confirmed in a separate Chinese cohort study using a TaqMan based genotyping assay (Guo X., Zhang Y., Li J. et al., Strong influence of human leukocyte antigen (HLA)-DP gene variants on development of persistent chronic hepatitis B virus carriers in the Han Chinese population, Hepatology 2011; 53: 422-8). Furthermore, a separate GWAS and replication analysis concluded similar results that there is significant association between the HLA-DP locus and the protective effects against persistent HBV infection in Japanese and Korean populations (Nishida N., Sawai H., Matsuura K. et al., Genome-wide association study confirming association of HLA-DP with protection against chronic hepatitis B and viral clearance in Japanese and Korean. PLos One 2012; 7: e39175). Finally, two additional SNPs (rs2856718 and rs7453920) within the HLA-DQ locus were found to have an independent effect of HLA-DQ variants on CHB susceptibility (Mbarek H., Ochi H., Urabe Y. et al., A genome-wide association study of chronic hepatitis B identified novel risk locus in a Japanese population, Hum. Mol. Genet. 2011; 20: 3884-92). Taken together, robust genetic evidence suggests that in the Asian population, polymorphic variations at the HLA region contribute significantly to the progression of chronic hepatitis B following acute infection in Asian populations.

Meta-analysis of controlled HBV clinical trials has demonstrated that conventional IFN alfa- or pegylated IFN alfa (2a or 2b)-containing treatment facilitated significant HBsAg clearance or seroconversion in CHB patients over lamivudine regimens (Li W. C., Wang M. R., Kong L. B. et al., Peginterferon alpha-based therapy for chronic hepatitis B focusing on HBsAg clearance or seroconversion: a meta-analysis of controlled clinical trials, BMC Infect. Dis. 2011; 11: 165-177). However, despite the fact that Peg-IFN has been successfully used in the treatment of CHB, little is known regarding the relationship between treatment response and the impact of host factors at the level of single nucleotide polymorphisms (SNPs). Pegylated interferon alfa, in combination with ribavirin (RBV) has been successfully used in the treatment of chronic hepatitis C virus (HCV) infection. A major scientific finding in how HCV patients respond to Peg-IFN/RBV treatment is that via genome-wide association studies (GWAS), genetic polymorphisms around the gene IL28B on chromosome 19 are strongly associated with treatment outcome (Ge D., Fellay J., Thompson A. J. et al., Genetic variation in IL28B predicts hepatitis C treatment-induced viral clearance, Nature 2009; 461: 399-401; Tanaka Y., Nishida N., Sugiyama M. et al., Genome-wide association of IL28B with response to pegylated interferon-alpha and ribavirin therapy for chronic hepatitis C, Nat. Genet. 2009; 41: 1105-9; Suppiah V., Moldovan M., Ahlenstiel G. et al., IL28B is associated with response to chronic hepatitis C interferon-alpha and ribavirin therapy, Nat. Genet. 2009; 41: 1100-4). IL28B encoded protein is a type III IFN (IFN-λ3) and forms a cytokine gene cluster with IL28A and IL29 at the same chromosomal region. IL28B can be induced by viral infection and has antiviral activity. However, in CHB patients treated with Peg-IFN, there are limited and somewhat conflicting data on the association of specific SNPs (e.g. rs12989760, rs8099917, rs12980275) around IL28B region with treatment responses (Lampertico P., Vigano M., Cheroni C. et al., Genetic variation in IL28B polymorphism may predict HBsAg clearance in genotype D, HBeAg negative patients treated with interferon alfa, AASLD 2010; Mangia A., Santoro R., Mottola et al., Lack of association between IL28B variants and HBsAg clearance after interferon treatment, EASL 2011; de Niet A., Takkenberg R. B., Benayed R. et al., Genetic variation in IL28B and treatment outcome in HBeAg-positive and -negative chronic hepatitis B patients treated with Peg interferon alfa-2a and adefovir, Scand. J. Gastroenterol. 2012, 47: 475-81; Sonneveld M. J., Wong V. W., Woltman A. M. et al., Polymorphisms near IL28B and serologic response to peginterferon in HBeAg-positive patients with chronic hepatitis B, Gastroenterology 2012; 142: 513-520).

IL28B genotype predicts response to pegylated-interferon (peg-IFN)-based therapy in chronic hepatitis C. Holmes et al. investigated whether IL28B genotype is associated with peg-IFN treatment outcomes in a predominantly Asian CHB cohort. IL28B genotype was determined for 96 patients (Holmes et al., IL28B genotype is not useful for predicting treatment outcome in Asian chronic hepatitis B patients treated with pegylated interferon-alpha, J. Gastroenterol. Hepatol., 2013, 28(5): 861-6). 88% were Asian, 62% were HBeAg-positive and 13% were METAVIR stage F3-4. Median follow-up time was 39.3 months. The majority of patients carried the CC IL28B genotype (84%). IL28B genotype did not differ according to HBeAg status. The primary endpoints were achieved in 27% of HBeAg-positive and 61% of HBeAg-negative patients. There was no association between IL28B genotype and the primary endpoint in either group. Furthermore, there was no difference in HBeAg loss alone, HBsAg loss, ALT normalisation or on-treatment HBV DNA levels according to IL28B genotype.

With whole blood sample collection in CHB patients who have been treated with Peg-IFN and have definite clinical outcomes, it is well justified that mechanistically understanding how host genetic factors affect treatment response and HBV disease biology will be tremendously beneficial to the future clinical practice of identifying patients who are likely to respond to Peg-IFN treatment and to the development of new HBV medicines.

### SUMMARY OF THE INVENTION

The present invention provides for methods for identifying patients who will respond to an anti-HBV treatment with anti-HBV agents, such as an interferon.

One embodiment of the invention provides methods of identifying a patient who may benefit from treatment with an anti-HBV therapy comprising an interferon, the methods comprising: determining the presence of a single nucleotide polymorphism in gene CADPS on chromosome 3 in a sample obtained from the patient, wherein the presence of at least one A allele at rs7633796 indicates that the patient may benefit from the treatment with the anti-HBV treatment.

A further embodiment of the inventions provides methods of predicting responsiveness of a patient suffering from an HBV infection to treatment with an anti-HBV treatment comprising an interferon, the methods comprising: determining the presence of a single nucleotide polymorphism in gene CADPS on chromosome 3 in a sample obtained from the patient, wherein the presence of at least one A allele at rs7633796 indicates that the patient is more likely to be responsive to treatment with the anti-HBV treatment.

Yet another embodiment of the invention provides methods for determining the likelihood that a patient with an HBV infection will exhibit benefit from an anti-HBV treatment comprising an interferon, the methods comprising: determining the presence of a single nucleotide polymorphism in gene CADPS on chromosome 3 in a sample obtained from the patient, wherein the presence of at least one A allele at rs7633796 indicates that the patient has increased likelihood of benefit from the anti-HBV treatment.

Even another embodiment of the invention provides methods for optimizing the therapeutic efficacy of an anti-HBV treatment comprising an interferon, the methods comprising: determining the presence of a single nucleotide polymorphism in gene CADPS on chromosome 3 in a sample obtained from the patient, wherein the presence of at least one A allele at rs7633796 indicates that the patient has increased likelihood of benefit from the anti-HBV treatment.

A further embodiment of the invention provides methods for treating an HBV infection in a patient, the methods comprising: (i) determining the presence of at least one A allele at rs7633796 in gene CADPS on chromosome 3 in a sample obtained from the patient and (ii) administering an effective amount of an anti-HBV treatment comprising an interferon to said patient, whereby the HBV infection is treated.

Yet another embodiment of the present invention provides methods for predicting HBeAg seroconversion and HBV DNA<2000 IU/ml at >=24-week follow-up of treatment (responders vs. non-responders) of an HBe-positive patient infected with HBV to interferon treatment comprising: (i) providing a sample from said human subject, detecting the presence of a single nucleotide polymorphism in gene CADPS on chromosome 3 and (ii) determining that said patient has a high response rate to interferon treatment measured as HBeAg seroconversion and HBV DNA<2000 IU/ml at >=24-week follow-up of treatment (responders vs. non-responders) if at least one A allele at rs7633796 is present.

Another embodiment of the invention provides methods of identifying a patient who may benefit from treatment with an anti-HBV therapy comprising an interferon, the methods comprising: determining the presence of a single nucleotide polymorphism in gene ARHGEF7 on chromosome 13 in a sample obtained from the patient, wherein the presence of at least one A allele at rs12584550 indicates that the patient may benefit from the treatment with the anti-HBV treatment.

A further embodiment of the inventions provides methods of predicting responsiveness of a patient suffering from an HBV infection to treatment with an anti-HBV treatment comprising an interferon, the methods comprising: determining the presence of a single nucleotide polymorphism in gene ARHGEF7 on chromosome 13 in a sample obtained from the patient, wherein the presence of at least one A allele at rs12584550 indicates that the patient is more likely to be responsive to treatment with the anti-HBV treatment.

Yet another embodiment of the invention provides methods for determining the likelihood that a patient with an HBV infection will exhibit benefit from an anti-HBV treatment comprising an interferon, the methods comprising: determining the presence of a single nucleotide polymorphism in gene ARHGEF7 on chromosome 13 in a sample obtained from the patient, wherein the presence of at least one A allele at rs12584550 indicates that the patient has increased likelihood of benefit from the anti-HBV treatment.

Even another embodiment of the invention provides methods for optimizing the therapeutic efficacy of an anti-HBV treatment comprising an interferon, the methods comprising: determining the presence of a single nucleotide polymorphism in gene ARHGEF7 on chromosome 13 in a sample obtained from the patient, wherein the presence of at least one A allele at rs12584550 indicates that the patient has increased likelihood of benefit from the anti-HBV treatment.

A further embodiment of the invention provides methods for treating an HBV infection in a patient, the methods comprising: (i) determining the presence of at least one A allele at rs12584550 in gene ARHGEF7 on chromosome 13 in a sample obtained from the patient and (ii) administering an effective amount of an anti-HBV treatment comprising an interferon to said patient, whereby the HBV infection is treated.

Yet another embodiment of the present invention provides methods for predicting HBeAg seroconversion and HBV DNA<2000 IU/ml at >=24-week follow-up of treatment (responders vs. non-responders) of an HBe-positive patient infected with HBV to interferon treatment comprising: (i) providing a sample from said human subject, detecting the presence of a single nucleotide polymorphism in gene ARHGEF7 on chromosome 13 and (ii) determining that said patient has a high response rate to interferon treatment measured as HBeAg seroconversion and HBV DNA<2000 IU/ml at >=24-week follow-up of treatment (responders vs. non-responders) if at least one A allele at rs12584550 is present.

Another embodiment of the invention provides methods of identifying a patient who may benefit from treatment with an anti-HBV therapy comprising an interferon, the methods comprising: determining the presence of a single nucleotide polymorphism in gene DOCK1 on chromosome 10 in a sample obtained from the patient, wherein the presence of at least one C allele at rs10765101 indicates that the patient may benefit from the treatment with the anti-HBV treatment.

A further embodiment of the inventions provides methods of predicting responsiveness of a patient suffering from an HBV infection to treatment with an anti-HBV treatment comprising an interferon, the methods comprising: determining the presence of a single nucleotide polymorphism in gene DOCK1 on chromosome 10 in a sample obtained from the patient, wherein the presence of at least one C allele at rs10765101 indicates that the patient is more likely to be responsive to treatment with the anti-HBV treatment.

Yet another embodiment of the invention provides methods for determining the likelihood that a patient with an HBV infection will exhibit benefit from an anti-HBV treatment comprising an interferon, the methods comprising: determining the presence of a single nucleotide polymorphism in gene DOCK1 on chromosome 10 in a sample obtained from the patient, wherein the presence of at least one C allele at rs10765101 indicates that the patient has increased likelihood of benefit from the anti-HBV treatment.

Even another embodiment of the invention provides methods for optimizing the therapeutic efficacy of an anti-HBV treatment comprising an interferon, the methods comprising: determining the presence of a single nucleotide polymorphism in gene DOCK1 on chromosome 10 in a sample obtained from the patient, wherein the presence of at least one C allele at rs10765101 indicates that the patient has increased likelihood of benefit from the anti-HBV treatment.

A further embodiment of the invention provides methods for treating an HBV infection in a patient, the methods comprising: (i) determining the presence of at least one C allele at rs10765101 in gene DOCK1 on chromosome 10 in a sample obtained from the patient and (ii) administering an effective amount of an anti-HBV treatment comprising an interferon to said patient, whereby the HBV infection is treated.

Yet another embodiment of the present invention provides methods for predicting HBeAg seroconversion and HBV DNA<2000 IU/ml at >=24-week follow-up of treatment (responders vs. non-responders) of an HBe-positive patient infected with HBV to interferon treatment comprising: (i) providing a sample from said human subject, detecting the presence of a single nucleotide polymorphism in gene DOCK1 on chromosome 10 and (ii) determining that said patient has a high response rate to interferon treatment measured as HBeAg seroconversion and HBV DNA<2000 IU/ml at >=24-week follow-up of treatment (responders vs. non-responders) if at least one C allele at rs10765101 is present.

Another embodiment of the invention provides methods of identifying a patient who may benefit from treatment with an anti-HBV therapy comprising an interferon, the methods comprising: determining the presence of a single nucleotide polymorphism in gene SYNJ1 on chromosome 21 in a sample obtained from the patient, wherein the presence of at least one G allele at rs10470165 indicates that the patient may benefit from the treatment with the anti-HBV treatment.

A further embodiment of the inventions provides methods of predicting responsiveness of a patient suffering from an HBV infection to treatment with an anti-HBV treatment comprising an interferon, the methods comprising: determining the presence of a single nucleotide polymorphism in gene SYNJ1 on chromosome 21 in a sample obtained from the patient, wherein the presence of at least one G allele at rs10470165 indicates that the patient is more likely to be responsive to treatment with the anti-HBV treatment.

Yet another embodiment of the invention provides methods for determining the likelihood that a patient with an HBV infection will exhibit benefit from an anti-HBV treatment comprising an interferon, the methods comprising: determining the presence of a single nucleotide polymorphism in gene SYNJ1 on chromosome 21 in a sample obtained from the patient, wherein the presence of at least one G allele at rs10470165 indicates that the patient has increased likelihood of benefit from the anti-HBV treatment.

Even another embodiment of the invention provides methods for optimizing the therapeutic efficacy of an anti-HBV treatment comprising an interferon, the methods comprising: determining the presence of a single nucleotide polymorphism in gene SYNJ1 on chromosome 21 in a sample obtained from the patient, wherein the presence of at least one G allele at rs10470165 indicates that the patient has increased likelihood of benefit from the anti-HBV treatment.

A further embodiment of the invention provides methods for treating an HBV infection in a patient, the methods comprising: (i) determining the presence of at least one G allele at rs10470165 in gene SYNJ1 on chromosome 21 in a sample obtained from the patient and (ii) administering an effective amount of an anti-HBV treatment comprising an interferon to said patient, whereby the HBV infection is treated.

Yet another embodiment of the present invention provides methods for predicting HBeAg seroconversion and HBV DNA<2000 IU/ml at >=24-week follow-up of treatment (responders vs. non-responders) of an HBe-positive patient infected with HBV to interferon treatment comprising: (i) providing a sample from said human subject, detecting the presence of a single nucleotide polymorphism in gene SYNJ1 on chromosome 21 and (ii) determining that said patient has a high response rate to interferon treatment measured as HBeAg seroconversion and HBV DNA<2000 IU/ml at >=24-week follow-up of treatment (responders vs. non-responders) if at least one G allele at rs10470165 is present.

Another embodiment of the invention provides methods of identifying a patient who may benefit from treatment with an anti-HBV therapy comprising an interferon, the methods comprising: determining the presence of a single nucleotide polymorphism in gene EGFR on chromosome 7 in a sample obtained from the patient, wherein the presence of at least one A allele at rs845562 indicates that the patient may benefit from the treatment with the anti-HBV treatment.

A further embodiment of the inventions provides methods of predicting responsiveness of a patient suffering from an HBV infection to treatment with an anti-HBV treatment comprising an interferon, the methods comprising: determining the presence of a single nucleotide polymorphism in gene EGFR on chromosome 7 in a sample obtained from the patient, wherein the presence of at least one A allele at rs845562 indicates that the patient is more likely to be responsive to treatment with the anti-HBV treatment.

Yet another embodiment of the invention provides methods for determining the likelihood that a patient with an HBV infection will exhibit benefit from an anti-HBV treatment comprising an interferon, the methods comprising: determining the presence of a single nucleotide polymorphism in gene EGFR on chromosome 7 in a sample obtained from the patient, wherein the presence of at least one A allele at rs845562 indicates that the patient has increased likelihood of benefit from the anti-HBV treatment.

Even another embodiment of the invention provides methods for optimizing the therapeutic efficacy of an anti-HBV treatment comprising an interferon, the methods comprising: determining the presence of a single nucleotide polymorphism in gene EGFR on chromosome 7 in a sample obtained from the patient, wherein the presence of at least one A allele at rs845562 indicates that the patient has increased likelihood of benefit from the anti-HBV treatment.

A further embodiment of the invention provides methods for treating an HBV infection in a patient, the methods comprising: (i) determining the presence of at least one A allele at rs845562 in gene EGFR on chromosome 7 in a sample obtained from the patient and (ii) administering an effective amount of an anti-HBV treatment comprising an interferon to said patient, whereby the HBV infection is treated.

Yet another embodiment of the present invention provides methods for predicting HBeAg seroconversion and HBV DNA<2000 IU/ml at >=24-week follow-up of treatment (responders vs. non-responders) of an HBe-positive patient infected with HBV to interferon treatment comprising: (i) providing a sample from said human subject, detecting the presence of a single nucleotide polymorphism in gene EGFR on chromosome 7 and (ii) determining that said patient has a high response rate to interferon treatment measured as HBeAg seroconversion and HBV DNA<2000 IU/ml at >=24-week follow-up of treatment (responders vs. non-responders) if at least one A allele at rs845562 is present.

Another embodiment of the invention provides methods of identifying a patient who may benefit from treatment with an anti-HBV therapy comprising an interferon, the methods comprising: determining the presence of a single nucleotide polymorphism in gene HSPG2 on chromosome 1 in a sample obtained from the patient, wherein the presence of at least one G allele (major allele) at rs4654771 indicates that the patient may benefit from the treatment with the anti-HBV treatment.

A further embodiment of the inventions provides methods of predicting responsiveness of a patient suffering from an HBV infection to treatment with an anti-HBV treatment comprising an interferon, the methods comprising: determining the presence of a single nucleotide polymorphism in gene HSPG2 on chromosome 1 in a sample obtained from the patient, wherein the presence of at least one G allele (major allele) at rs4654771 indicates that the patient is more likely to be responsive to treatment with the anti-HBV treatment.

Yet another embodiment of the invention provides methods for determining the likelihood that a patient with an HBV infection will exhibit benefit from an anti-HBV treatment comprising an interferon, the methods comprising: determining the presence of a single nucleotide polymorphism in gene HSPG2 on chromosome 1 in a sample obtained from the patient, wherein the presence of at least one G allele (major allele) at rs4654771 indicates that the patient has increased likelihood of benefit from the anti-HBV treatment.

Even another embodiment of the invention provides methods for optimizing the therapeutic efficacy of an anti-HBV treatment comprising an interferon, the methods comprising: determining the presence of a single nucleotide polymorphism in gene HSPG2 on chromosome 1 in a sample obtained from the patient, wherein the presence of at least one G allele (major allele) at rs4654771 indicates that the patient has increased likelihood of benefit from the anti-HBV treatment.

A further embodiment of the invention provides methods for treating an HBV infection in a patient, the methods comprising: (i) determining the presence of at least one G allele (major allele) at rs4654771 in gene HSPG2 on chromosome 1 in a sample obtained from the patient and (ii) administering an effective amount of an anti-HBV treatment comprising an interferon to said patient, whereby the HBV infection is treated.

Yet another embodiment of the present invention provides methods for predicting HBeAg seroconversion and HBV DNA<2000 IU/ml at >=24-week follow-up of treatment (responders vs. non-responders) of an HBe-positive patient infected with HBV to interferon treatment comprising: (i) providing a sample from said human subject, detecting the presence of a single nucleotide polymorphism in gene HSPG2 on chromosome 1 and (ii) determining that said patient has a high response rate to interferon treatment measured as HBeAg seroconversion and HBV DNA<2000 IU/ml at >=24-week follow-up of treatment (responders vs. non-responders) if at least one G allele (major allele) at rs4654771 is present.

Another embodiment of the present invention provides polymorphic signature that predicts responsiveness of a patient suffering from an HBV infection to treatment with an anti-HBV treatment comprising an interferon, said signature comprising the determination of the presence or absence of at least one the following single nucleotide polymorphisms (SNPs) rs2970471, rs4142734, rs10824875, rs9567867, rs2542943, rs604241, rs4899150, rs508636, rs12626242, rs7633796, rs7947950, rs12584550, rs10765101, rs12435908, rs845023, rs12627478, rs10470165, rs1867475, rs9856296, rs6776709, rs4684175, rs4685060, rs4539348, rs13297144, rs11072478, rs260010, rs13395925, rs845562, rs12568559, rs10193128, exm680251, rs4840946, rs4840947, exm680207, rs9288685, rs9322467, rs6733352, rs7633147, rs943172, rs4348723, rs6428677, rs4654771, rs2500499, rs4840939, rs10494323, exm-rs4133289, exm-rs10489849, exm114297, rs10489849, rs17403692, rs2662605, rs17094430, rs11110982, rs11177673, and rs736452, wherein the presence of at least one of these SNPs predicts responsiveness of a patient suffering from an HBV infection to treatment with an anti-HBV treatment comprising an interferon.

In some embodiments, the interferon is selected from the group of peginterferon alfa-2a, peginterferon alfa-2b, interferon alfa-2a and interferon alfa-2b.

In some embodiments, the interferon is a peginterferon alfa-2a conjugate having the formula:

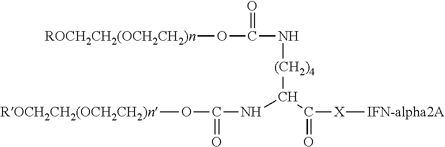

wherein R and R′ are methyl, X is NH, and n and n′ are individually or both either 420 or 520.

### BRIEF DESCRIPTION OF THE DRAWINGS

FIG. 1: Bar chart of the number of markers by chromosome in the GWAS Marker Set. Of 925,371 markers, 1,003 markers were not plotted due to unknown genomic location.

FIG. 2: Scree plot for ancestry analysis.

FIG. 3: The first two principal components of ancestry for HapMap individuals only. Population codes are as listed in Table 3.

FIG. 4: The first two principal components of ancestry for HapMap individuals ; coloured according to population group (Table 3). Overlaid are patients who will be incorporated into PGx-CN-Interim1 (black crosses) and those that will be incorporated into PGx-non-CN-Interim1 (grey crosses).

FIGS. 5A, 5B, and 5C: Manhattan Plots for Endpoint 1

FIGS. 6A, 6B, and 6C: QQ Plots for Endpoint 1

FIGS. 7A, 7B, and 7C: Manhattan Plots for Endpoint 2

FIGS. 8A, 8B, and 8C: QQ Plots for Endpoint 2

FIGS. 9A, 9B, and 9C: Manhattan Plots for Endpoint 3

FIGS. 10A, 10B, and 10C: QQ Plots for Endpoint 3

FIGS. 11A, 11B, and 11C: Manhattan Plots for Endpoint 4

FIGS. 12A, 12B, and 12C: QQ Plots for Endpoint 4

FIGS. 13A, 13B, and 13C: Manhattan Plots for Endpoint 5

FIGS. 14A, 14B, and 14C: QQ Plots for Endpoint 5

FIGS. 15A, 15B, and 15C: Manhattan Plots for Endpoint 6

FIGS. 16A, 16B, and 16C: QQ Plots for Endpoint 6

FIG. 17: Interactions among genes associated at the suggestive level with response to Pegasys

### DETAILED DESCRIPTION OF THE INVENTION

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The terms “sample” or “biological sample” refers to a sample of tissue or fluid isolated from an individual, including, but not limited to, for example, tissue biopsy, plasma, serum, whole blood, spinal fluid, lymph fluid, the external sections of the skin, respiratory, intestinal and genitourinary tracts, tears, saliva, milk, blood cells, tumors, organs. Also included are samples of in vitro cell culture constituents (including, but not limited to, conditioned medium resulting from the growth of cells in culture medium, putatively virally infected cells, recombinant cells, and cell components).

The terms “interferon” and “interferon-alpha” are used herein interchangeably and refer to the family of highly homologous species-specific proteins that inhibit viral replication and cellular proliferation and modulate immune response. Typical suitable interferons include, but are not limited to, recombinant interferon alpha-2b such as Intron® A interferon available from Schering Corporation, Kenilworth, N.J., recombinant interferon alpha-2a such as Roferon®-A interferon available from Hoffmann-La Roche, Nutley, N.J., recombinant interferon alpha-2C such as Berofor® alpha 2 interferon available from Boehringer Ingelheim Pharmaceutical, Inc., Ridgefield, Conn., interferon alpha-n1, a purified blend of natural alpha interferons such as Sumiferon® available from Sumitomo, Japan or as Wellferon® interferon alpha-n1 (INS) available from the Glaxo-Wellcome Ltd., London, Great Britain, or a consensus alpha interferon such as those described in U.S. Pat. Nos. 4,897,471 and 4,695,623 (especially Examples 7, 8 or 9 thereof) and the specific product available from Amgen, Inc., Newbury Park, Calif., or interferon alpha-n3 a mixture of natural alpha interferons made by Interferon Sciences and available from the Purdue Frederick Co., Norwalk, Conn., under the Alferon Tradename. The use of interferon alpha-2a or alpha-2b is preferred. Interferons can include pegylated interferons as defined below.

The terms “pegylated interferon”, “pegylated interferon alpha” and “peginterferon” are used herein interchangeably and means polyethylene glycol modified conjugates of interferon alpha, preferably interferon alfa-2a and alfa-2b. Typical suitable pegylated interferon alpha include, but are not limited to, Pegasys® and Peg-Intron®.

As used herein, the terms “allele” and “allelic variant” refer to alternative forms of a gene including introns, exons, intron/exon junctions and 3′ and/or 5′ untranslated regions that are associated with a gene or portions thereof. Generally, alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides.

As used herein, the term “polymorphism” refers to the coexistence of more than one form of a nucleic acid, including exons and introns, or portion (e.g., allelic variant) thereof. A portion of a gene of which there are at least two different forms, i.e., two different nucleotide sequences, is referred to as a polymorphic region of a gene. A polymorphic region can be a single nucleotide, i.e. “single nucleotide polymorphism” or “SNP”, the identity of which differs in different alleles. A polymorphic region can also be several nucleotides long.

Numerous methods for the detection of polymorphisms are known and may be used in conjunction with the present invention. Generally, these include the identification of one or more mutations in the underlying nucleic acid sequence either directly (e.g., in situ hybridization) or indirectly (identifying changes to a secondary molecule, e.g., protein sequence or protein binding).

One well-known method for detecting polymorphisms is allele specific hybridization using probes overlapping the mutation or polymorphic site and having about 5, 10, 20, 25, or 30 nucleotides around the mutation or polymorphic region. For use in a kit, e.g., several probes capable of hybridizing specifically to allelic variants, such as single nucleotide polymorphisms, are provided for the user or even attached to a solid phase support, e.g., a bead or chip.

### Abbreviations

| | |
|---|---|
| AIC | Akaike Information Criterion |
| ALT | Alanine aminotransferase |
| Anti-HBs | Antibody to hepatitis B surface antigen |
| DNA | Deoxyribonucleic acid |
| GWAS | Genome-wide Association Study |
| HAV | Hepatitis A Virus |
| HBe | Hepatitis B ‘e’ Antigen |
| HBeAg | Hepatitis B ‘e’ Antigen |
| HBV | Hepatitis B Virus |
| HCV | Hepatitis C Virus |
| HIV | Human Immunodeficiency Virus |
| HLA | Human Leucocyte Antigen |
| HWE | Hardy-Weinberg Equilibrium |
| IU/ml | International units per milliliter |
| PCA | Principal Components Analysis |
| PEGASYS | Pegylated Interferon alpha 2a 40 KD; Peg-IFN |
| Peg-IFN | Pegylated Interferon alpha 2a 40 KD; PEGASYS |
| QC | Quality Checks |
| qHBsAg | Quantitative Hepatitis B Surface Antigen |
| S-loss | Surface Antigen Loss |
| SNP | Single Nucleotide Polymorphism |
| SPC | Summary of Product Characteristics |
| TLR | Toll-like Receptor |
| Tx | Treatment |
| Vs. | Versus |

### EXAMPLES

### Objectives and Endpoints

The objective was to determine genetic variants associated with response to treatment with PEGASYS-containing regimen in patients with Chronic Hepatitis B.

The following primary endpoints were considered:
1. HBe-positive patients: E-seroconversion or S-loss at >=24-week follow-up
2. HBe-positive patients: (E-seroconversion plus HBV DNA<2000 IU/ml) or S-loss at >=24-week follow-up
3. HBe-negative patients: HBV DNA<2000 IU/ml or S-loss at >=24-week follow-up
4. E-seroconversion or S-loss at >=24-week follow-up if HBe-positive and HBV DNA<2000 IU/ml or S-loss at >=24-week follow-up if HBe-negative (1 and 3)
5. (E-seroconversion plus HBV DNA<2000 IU/ml) or S-loss at >=24-week follow-up if HBe-positive and HBV DNA<2000 IU/ml or S-loss at >=24-week follow-up if HBe-negative (2 and 3)
6. S-loss at >=24-week follow-up For all endpoints and all markers, the null hypothesis of no association, between the genotype and the endpoint, was tested against the two-sided alternative that association exists.

### Study Design

A cumulative meta-analysis, of data from company-sponsored clinical trials, and data from patients in General Practice care, is in progress. The combined data will, at the final analysis, comprise up to 1500 patients who have been treated with Pegasys for at least 24 weeks, with or without a nucleotide/nucleoside analogue, and with 24 weeks of follow-up data available.

The following trials/patient sources were considered for inclusion:
RGT (ML22266)
S-Collate (MV22009)
SoN (MV22430)
Switch (ML22265)
Combo
New Switch (ML27928)
NEED
Italian cohort of PEG.Be.Liver
Professor Teerha (Thailand): clinical practice patients and some legacy Ph3 patients
Professor Hongfei Zhang (Beijing, China): clinical practice patients and some legacy Ph3 patients
Professor Yao Xie (Beijing, China): clinical practice patients
Professor Xin Yue Chen (Beijing, China): clinical practice patients

Adult patients with chronic hepatitis B (male or female patients>18 years of age) must meet the following criteria for study entry:
Previously enrolled in a Roche study and treated for chronic hepatitis B for at least 24 weeks with Peg-IFN±nucleoside analogue (lamivudine or entacavir) or Peg-IFN±nucleotide analogue (adefovir) with >24-week post-treatment follow-up or;
Treated in general practice for chronic hepatitis B with Peg-IFN according to standard of care and in line with the current summary of product characteristics (SPC)/local labeling who have no contra-indication to Peg-IFN therapy as per the local label and have been treated with Peg-IFN for at least 24 weeks and have >24-week post-treatment response available at the time of blood collection.
Patients are not infected with HAV, HCV, or HIV
Patients should have the following medical record available (either from historical/ongoing study databases or from medical practice notes):
   Demographics (e.g. age, gender, ethnic origin)
   Pre-therapy HBeAg status, known or unknown HBV genotype
   Quantitative HBV DNA by PCR Test in IU/ml over time (e.g. baseline, on-treatment: 12- and 24-week, post-treatment: 24-week)
   Quantitative HBsAg test (if not available, qualitative HBsAg test) and anti-HBs over time (e.g. baseline, on-treatment: 12- and 24-week, post-treatment: 24-week)
   Serum ALT over time (e.g. baseline, on-treatment: 12- and 24-week, post-treatment: 24-week)

It is noted that all patients will have received active regimen.

### Analysis Populations

The majority of patients will be from China. For the purposes of statistical analysis, four analysis populations were defined as follows:
PGx-FAS is all patients with at least one genotype
PGx-GT is the subset of PGx-FAS whose genetic data passes quality checks
PGx-CN is the subset of PGx-GT who share a common genetic background in the sense that they cluster with CHB and CHD reference subjects from HapMap version3 (see below)
PGx-non-CN is the remainder of PGx-GT who do not fall within PGx-CN

Additional suffices are appended as HBePos or HBeNeg for the HBe-Positive and HBe-Negative subsets respectively, and as interim1, . . . interim3, and final, according to the stage of the analysis.

### Genetic Markers

The GWAS marker panel was the Illumina OmniExpress Exome microarray (www.illumina.com), consisting of greater than 750,000 SNP markers and greater than 250,000 exonic markers. The group of markers which passed quality checks are referred to as the GWAS Marker Set.

### General Considerations for Data Analysis

The GWAS is hypothesis-free. Markers with unadjusted p<5×10⁻⁸ were considered to be genome-wide significant. In the interests of statistical power, no adjustment was made for multiple endpoints or multiple rounds of analysis.

### Demographic and Baseline Characteristics

Table 1 below shows a brief summary of the baseline and demographic characteristics of the 137 patients in PGx-FAS-interim1 and separately, of the 653 patients in current PGx-FAS-interim2. It was noted that members of the current interim tend to be older in age, and much less likely to self-report as ‘Oriental’, although a substantial number now self-report as ‘Asian’.

TABLE 1
Baseline and Demographic Characteristics
for PGx-FAS-interim1 and PGx-FAS-interim2

| Variable | Category | Statistics |
|---|---|---|
| Count (n) | | |
| Sex | Male | n (%) |
| | Female | n (%) |
| Age (yr) | | Mean (SE) |
| Race | Oriental | n (%) |
| | White/Caucasian | n (%) |
| | Asian | n (%) |
| | Other | n (%) |
| Height (cm) | | Mean (SE) |
| Weight (kg) | | Mean (SE) |
| BMI (kg/m\hat{ }2) | | Mean (SE) |
| Baseline ALT (U/L) | | Median (IQR) |

| Variable | PGx-FAS-interim1 | PGx-FAS-interim2 |
|---|---|---|
| Count (n) | 137 | 653 |
| Sex | 88 (64%) | 433 (66%) |
| | 49 (36%) | 220 (34%) |
| Age (yr) | 32.25 (0.848) | 38.19 (0.451) |
| Race | 119 (87%) | 270 (41%) |
| | 7 (5%) | 229 (35%) |
| | 0 (0%) | 112 (17%) |
| | 11 (8%) | 42 (6%) |
| Height (cm) | 168.26 (0.766) | 167.9 (0.342) |
| Weight (kg) | 67.74 (1.43) | 66.93 (0.597) |
| BMI (kg/m\hat{ }2) | 23.78 (0.416) | 23.58 (0.167) |
| Baseline ALT (U/L) | 123 (119) | 92 (104) |

### Analysis of Gentic Data

### Quality Checks by Patient

### Methods

The following criteria were assessed, on the basis of unfiltered GWAS data, in 653 patients of any self-reported race (PGx-FAS-Interim2).
<30% heterozygosity genome-wide
<5% missing genotype data
Reported sex consistent with X-chromosome data
<30% genotype-concordance with another sample

### Results

All patients displayed <30% heterozygosity genome-wide. Three patients namely, 4360, 8529 and 8076 had 5% or more missing genotypes. Two samples, attributed to 5076 and 8554, were expected to be female, but showed high levels of X-chromosome homozygosity. Two pairs namely, 6454 and 9850, and 9114 and 9180 were seen to be first-degree relative pairs so for each pair, the patient with a higher level of missing genotype data was excluded from consideration.

In this way, seven patients were excluded from further analysis; their details are provided in Table 2 below. The remaining 646 patients, whose genetic data satisfied the criteria above, were incorporated into the PGx-GT-Interim2 Set.

TABLE 2
Seven patients whose genetic data failed
quality checks; NA represents missing

| ANONID | PROTO | AGE | SEX | RACE |
|---|---|---|---|---|
| 4360 | GV28855 | 61 | MALE | WHITE |
| 8529 | GV28855 | 52 | MALE | WHITE |
| 8076 | MV22430 | 28 | MALE | ORIENTAL |
| 5076 | GV28855 | 45 | FEMALE | WHITE |
| 8554 | MV22430 | 35 | FEMALE | MAORI |
| 6454 | MV22430 | 55 | FEMALE | CAUCASIAN |
| 9114 | GV28855 | 32 | MALE | WHITE |

| ANONID | HGTCM | WGTKG | BMI | GENO | HBE_BS |
|---|---|---|---|---|---|
| 4360 | NA | NA | NA | A | POSITIVE |
| 8529 | NA | NA | NA | NA | NEGATIVE |
| 8076 | 175 | 65 | 21.22449 | B | POSITIVE |
| 5076 | NA | NA | NA | NA | NA |
| 8554 | 173 | 77 | 25.72756 | D | POSITIVE |
| 6454 | 170 | 98.9 | 34.22145 | B | POSITIVE |
| 9114 | NA | NA | NA | D | POSITIVE |

### Quality Checks by Marker

### Methods

Markers were assessed for missing data. Those with greater than 5% missing data were excluded from further analysis.

### Results

It was noted that all of the first interim data, and a subset of the second interim data, were derived from Human Omni Express Exome 8v1B, whereas the majority of the second interim data were derived from Human Omni Express Exome 8v1.2A.

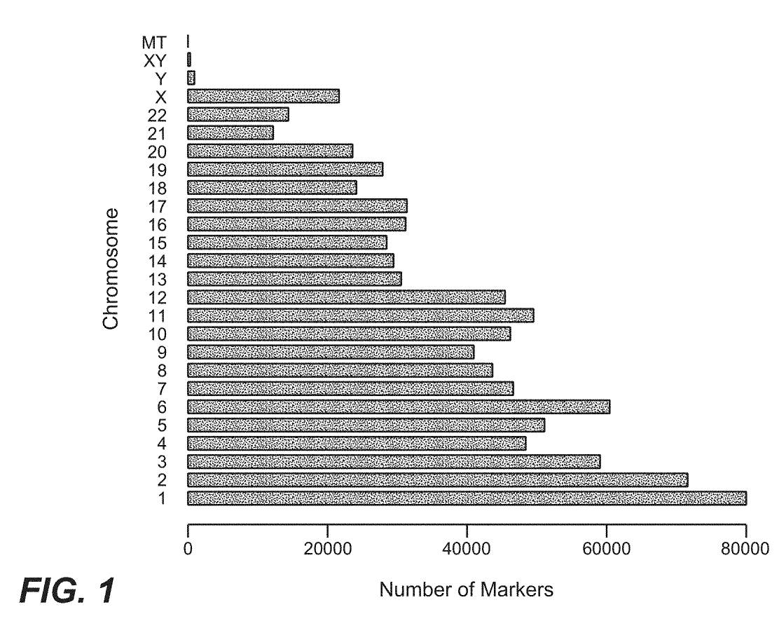

In order to perform the meta-analysis, the overlapping set of 925,371 markers, with <5% missing overall, was incorporated into the GWAS Marker Set. Their distribution by chromosome is shown in FIG. 1.

In the current interim analysis, markers were categorized as rare or non-rare, using a frequency threshold of 5%. In this way, a total of 323782 markers were considered rare; 601,589 were considered non-rare.

### Multivariate Analysis of Ancestry

Principal Components Analysis (PCA) is a technique for reducing the dimensionality of a data set. It linearly transforms a set of variables into a smaller set of uncorrelated variables representing most of the information in the original set (Dunteman, 1989). In the current study, the marker variables were transformed into principal components which were compared to self-reported ethnic groupings. The objective is, in preparation for association testing, to determine clusters of individuals who share a homogeneous genetic background.

A suitable set of 134,575 markers for ancestry analysis was obtained as described in statistical report for Interim Analysis 1. Of this set, 131,924 had at least 5% frequency in interim 2 data. PCA was therefore applied using 131,924 markers, genotyped across 646 study individuals and 988 HapMap reference individuals (Table 3).

TABLE 3
Details of the HapMap version 3 reference subjects

| Code | Description | Count |
|---|---|---|
| MKK | Maasai in Kinyawa, Kenya | 143 |
| LWK | Luhya in Webuye, Kenya | 90 |
| YRI | Yoruba in Ibadan, Nigeria | 113 |
| ASW | African ancestry in Southwest USA | 49 |
| CEU | Utah residents with Northern and Western | 112 |
| | European ancestry from the CEPH collection | |
| TSI | Tuscans in Italy | 88 |
| MEX | Mexican ancestry in Los Angeles, California | 50 |
| GIH | Gujarati Indians in Houston, Texas | 88 |
| JPT | Japanese in Tokyo, Japan | 86 |
| CHD | Chinese in Metropolitan Denver, Colorado | 85 |
| CHB | Han Chinese in Bejing, China | 84 |
| | TOTAL | 988 |

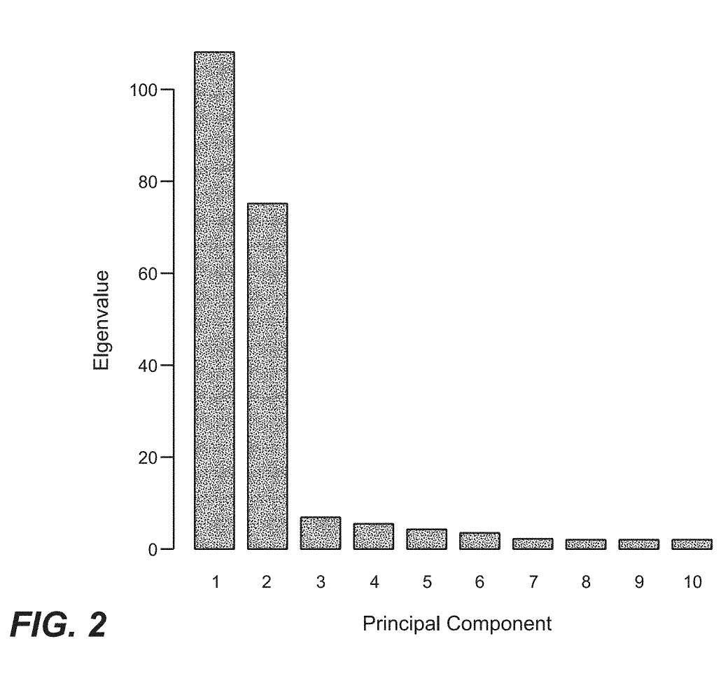

FIG. 2 shows the scree plot for the analysis. It is clear that the majority of information, indicated by the highest eigenvalues, was obtained from the first two principal components of ancestry, with little gain in information from subsequent components.

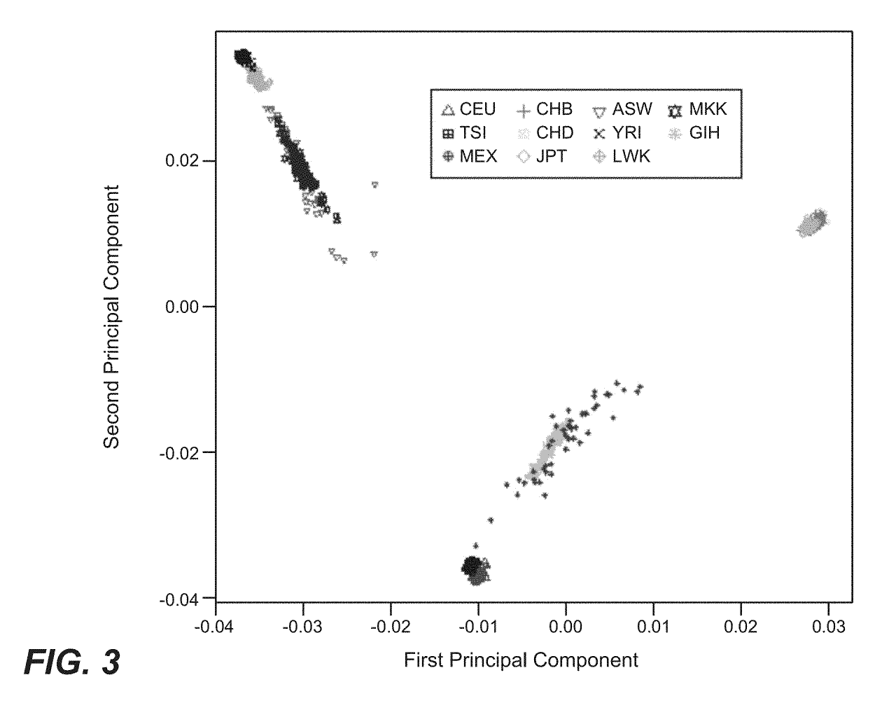

FIG. 3 shows the results of PCA for the HapMap reference data only. Four clusters are visible in this two-dimensional representation. Reading clockwise from top left, they are: African origin (blue/orange/pink/maroon), Southeast Asian (yellow/blue/green), Mexican (dark green) and South Asian Origin (grey), and Northern and Western European (blue/red).

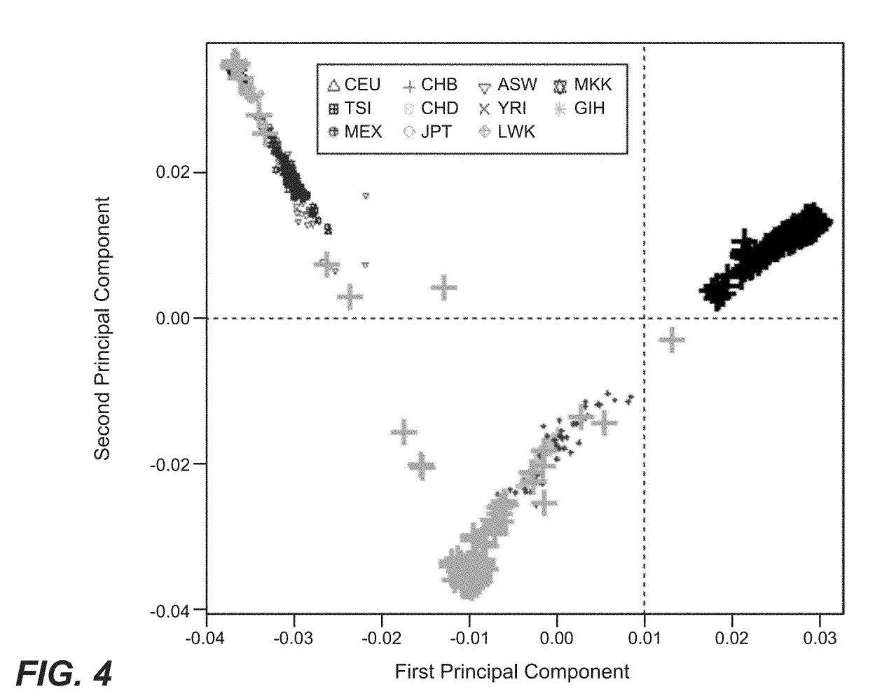

FIG. 4 shows the same data with study participants overlaid as crosses. Patients included in PGx-CN-Interim2 are given by black crosses; patients included in PGx-nonCN-Interim2 are given by grey crosses. As observed in the first interim analysis, the PGx-CN-Interim2 study participants represent a genetically more diverse group of individuals than the reference set. The study participants are likely to have been drawn from different countries in South-East Asia.

For the purposes of genetic analysis, PGx-CN-Interim2 was therefore made up of the 390 patients falling in a cluster around the Chinese and Japanese reference individuals. A total of 256 patients, whose plotted ancestry clearly departed from that cluster, made up PGx-non-CN-Interim2.

The number of patients in each planned analysis is given in Table 4 below. As stated earlier, the six endpoints are numbered as follows:
1. HBe-positive patients: E-seroconversion or S-loss at >=24-week follow-up
2. HBe-positive patients: (E-seroconversion plus HBV DNA<2000 IU/ml) or S-loss at >=24-week follow-up
3. HBe-negative patients: HBV DNA<2000 IU/ml or S-loss at >=24-week follow-up
4. E-seroconversion or S-loss at >=24-week follow-up if HBe-positive and HBV DNA<2000 IU/ml or S-loss at >=24-week follow-up if HBe-negative (1 and 3)
5. (E-seroconversion plus HBV DNA<2000 IU/ml) or S-loss at >=24-week follow-up if HBe-positive and HBV DNA<2000 IU/ml or S-loss at >=24-week follow-up if HBe-negative (2 and 3)
6. S-loss at >=24-week follow-up

It is noted that 24 patients did not have HBe data, so their response, as defined by endpoints 1-5, could not be determined. Furthermore, six of the analyses (each to be performed under two assumed modes of inheritance) contained at least one group with fewer than 30 patients, and so were not expected to be informative.

### Assessment of Covariates

In order to determine the covariates for the genome-wide association analysis, a series of variables were tested for association with each endpoint, using backwards stepwise regression. In accordance with the planned association analysis, the subject set for Endpoints 1, and 2 was PGx-GT-HBe-Pos-Interim2 (n=391); the subject set for Endpoint 3 was PGx-GT-HBe-Neg-Interim2 (n=231) and the subject set for endpoints 4, 5 and 6 was all members of PGx-GT-Interim2 (n=646). Backwards steps were taken on the basis of the Akaike Information Criterion (AIC).

The covariates in the full model were as follows: Age, Sex, Baseline HBV DNA, Baseline ALT, HBV genotype, Concomitant use of nucleotide/ nucleoside analogues, and Study. Principal Components of Ancestry were included for Endpoints 4 and 5, due to inclusion of both HBe-positive and HBe-negative groups, together with reasonable responder counts. Baseline HBV and Baseline ALT were both log-transformed in order to improve symmetry.

Tables 5-10 show the covariates selected for Endpoints 1-6. It can be seen that baseline HBV DNA and baseline ALT were each selected in five out of six models.

TABLE 5
Covariates selected by backwards stepwise
regression for Endpoint 1

| Variable | Odds Ratio | 95% CI | P-value |
|---|---|---|---|
| (Intercept) | 1.23 | 0.16-9.56 | 0.8446 |
| LOGDNA0 | 0.80 | 0.73-0.88 | <0.0001 |
| LOGALT0 | 2.05 | 1.46-2.89 | <0.0001 |
| NUCLO_N | 0.33 | 0.16-0.66 | 0.0019 |
| ML21827 | 0.36 | 0.14-0.96 | 0.0422 |
| MV22430 | 0.62 | 0.28-1.37 | 0.2360 |

TABLE 6
Covariates selected by backwards stepwise
regression for Endpoint 2

| Variable | Odds Ratio | 95% CI | P-value |
|---|---|---|---|
| (Intercept) | 1.35 | 0.14-13.36 | 0.7992 |
| LOGDNA0 | 0.81 | 0.73-0.90 | <0.0001 |
| LOGALT0 | 1.96 | 1.34-2.86 | 0.0005 |
| NUCLO_N | 0.26 | 0.10-0.67 | 0.0051 |
| ML21827 | 0.16 | 0.05-0.54 | 0.0031 |
| MV22430 | 0.30 | 0.11-0.81 | 0.0175 |

TABLE 7
Covariates selected by backwards stepwise
regression for Endpoint 3

| Variable | Odds Ratio | 95% CI | P-value |
|---|---|---|---|
| (Intercept) | 9.40 | 3.61-24.47 | <0.0001 |
| LOGDNA0 | 0.84 | 0.77-0.91 | <0.0001 |

TABLE 8
Covariates selected by backwards stepwise
regression for Endpoint 4

| Variable | Odds Ratio | 95% CI | P-value |
|---|---|---|---|
| (Intercept) | 14.86 | 3.96-55.69 | <0.0001 |
| LOGDNA0 | 0.76 | 0.71-0.82 | <0.0001 |
| LOGALT0 | 1.64 | 1.26-2.14 | 0.0002 |
| GENO_D | 0.64 | 0.35-1.19 | 0.1584 |
| NUCLO_N | 0.28 | 0.13-0.56 | 0.0004 |
| PC3 | 0.00 | 0.00-0.00 | 0.0020 |
| ML21827 | 0.24 | 0.10-0.59 | 0.0020 |
| MV22430 | 0.40 | 0.19-0.83 | 0.0145 |

TABLE 9
Covariates selected by backwards stepwise
regression for Endpoint 5

| Variable | Odds Ratio | 95% CI | P-value |
|---|---|---|---|
| (Intercept) | 26.04 | 5.71-118.84 | <0.0001 |
| LOGDNA0 | 0.76 | 0.71-0.81 | <0.0001 |
| LOGALT0 | 1.54 | 1.16-2.04 | 0.0025 |
| GENO_D | 0.62 | 0.33-1.17 | 0.1391 |
| NUCLO_N | 0.21 | 0.08-0.54 | 0.0013 |
| PC3 | 0 | 0-0 | 0.0024 |
| ML21827 | 0.09 | 0.03-0.30 | <0.0001 |
| MV22430 | 0.18 | 0.07-0.45 | 0.0003 |

TABLE 10
Covariates selected by backwards stepwise
regression for Endpoint 6

| Variable | Odds Ratio | 95% CI | P-value |
|---|---|---|---|
| (Intercept) | 0.15 | 0.01-2.10 | 0.1571 |
| AGE | 1.04 | 1.00-1.07 | 0.0365 |
| LOGALT0 | 0.55 | 0.34-0.91 | 0.0197 |

### Univariate Association Analysis

### Methods

Due to the modest group counts in the current interim analysis, markers were excluded from single-point association analysis if they had frequency less than 5%. The remaining 601,589 markers were coded in two ways as follows. Firstly they were coded according to an additive model, given by the count of the number of minor alleles. Secondly they were coded according to a dominant model of inheritance, based upon carriage of the minor allele.

Thirty-six rounds of association analysis were conducted due to three patient sets and six endpoints, each under two modes of inheritance. The following model was fitted using multivariate logistic regression:

### Endpoint=Intercept+[Covariates]+Marker

Covariates were applied as selected above (Section 8.4). In addition, adjustments for study were applied in all analyses, and adjustments for first two principal components of ancestry were applied in analyses of subsets of PGx-GT-Interim2 and PGx-nonCN-Interim2.

The significance of each marker was determined using a t-test. The genomic control lambda was calculated for each GWAS analysis and QQ-plots were examined, but no clear evidence of test-statistic inflation was found (Devlin and Roeder 1999). Maximum lambda was 1.06.

All markers were tested, using a chi-square test, for departure from Hardy-Weinberg Equilibrium (HWE) in PGx-GT-Interim2, PGx-nonCN-Interim2 and PGx-CN-Interim2. The results were used to assist in the interpretation of association analysis output. In the tabulated results below, both the minor allele frequency (MAF) and the Hardy-Weinberg result are shown for the relevant, ancestry-defined patient-group.

### Results for Endpoint 1

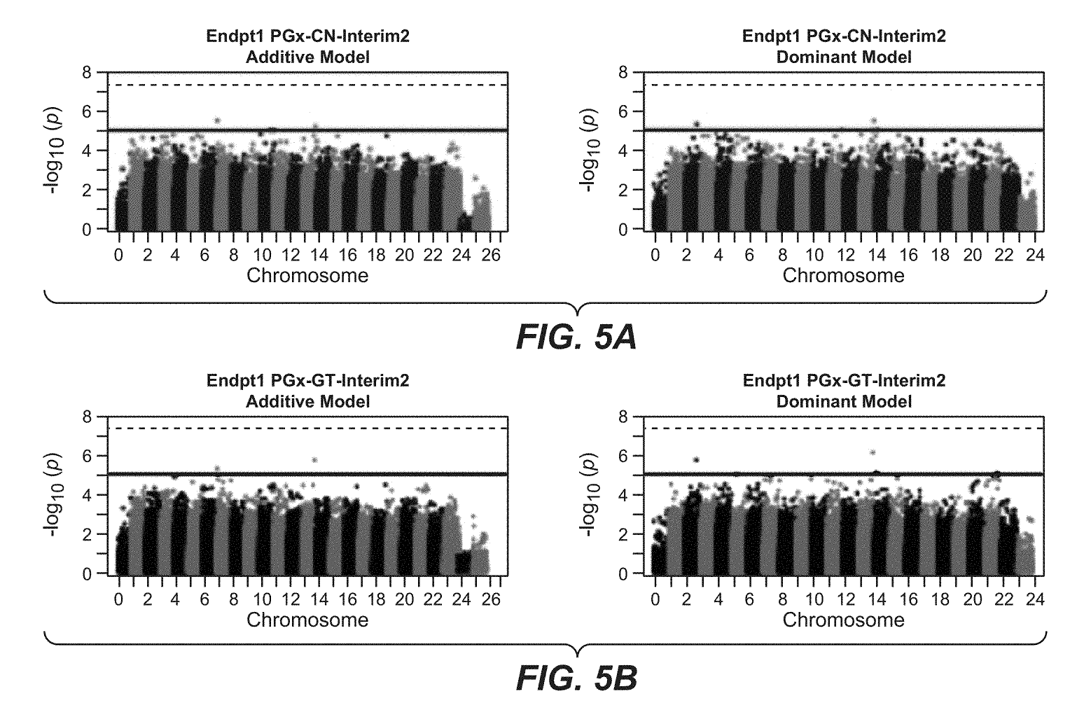

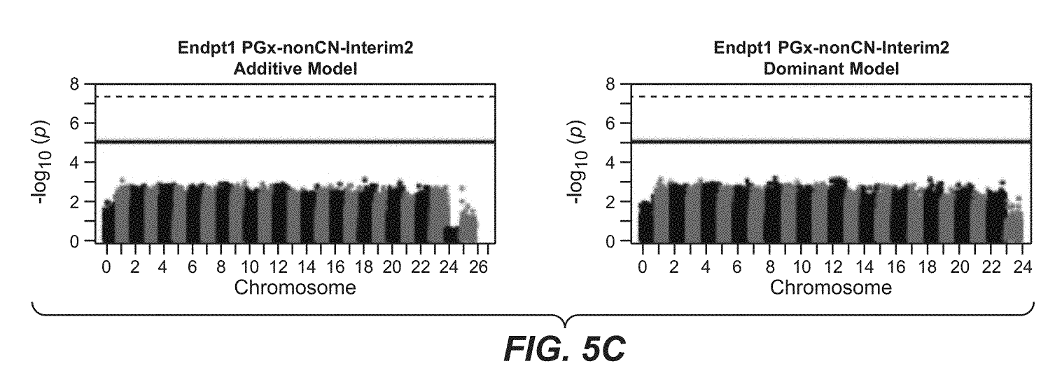

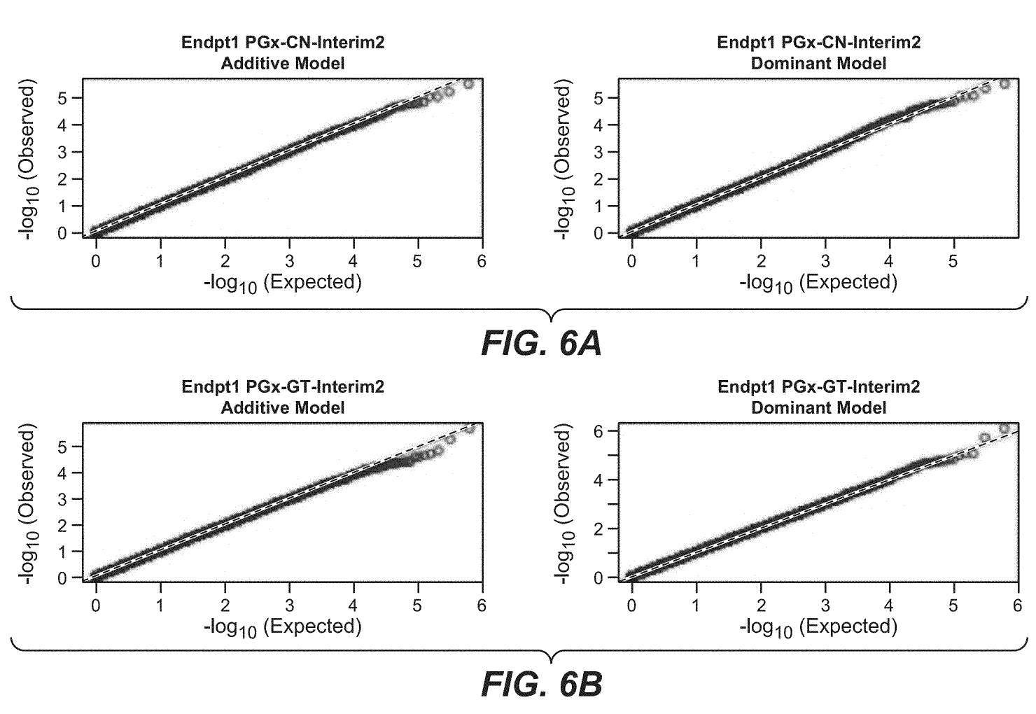

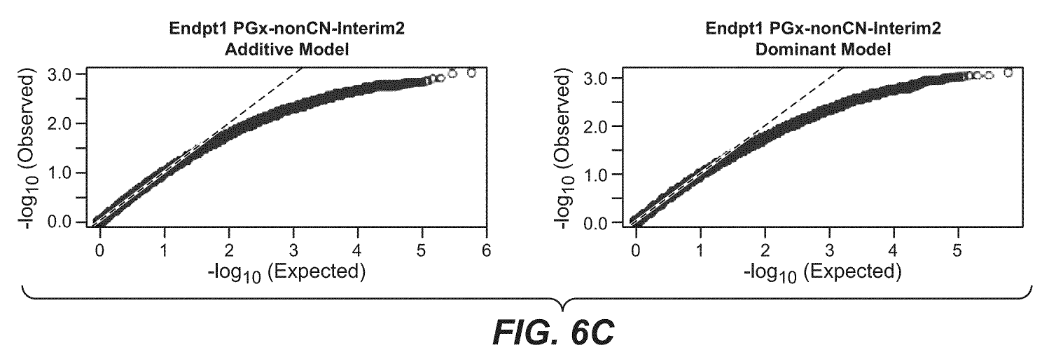

FIGS. 5 and 6 show the Manhattan plots and QQ plots respectively, for Endpoint 1. The first four QQ-plots are seen to track the 45-degree line, indicating that the p-value distributions are approximately as expected by chance. The QQ-plots for PGx-nonCN-HBe-Pos-Interim2 both dip below the 45-degree line, indicating reduced statistical power; the final two Manhattan plots are correspondingly flat. It was noted that there were only 12 responders in these last two analyses.

Details of markers with p<10⁻⁵ are given in Tables 11-14. No marker had p<10⁻⁵ in PGx-nonCN-HBe-Pos-Interim2, under either mode of inheritance.

TABLE 11
Association Results with p < 10⁻⁵ for Endpoint
1 in PGx-CN-HBe-Pos-Interim2, additive model

| Chr | SNP | BP | HWE(p) | MAF |
|---|---|---|---|---|
| 7 | rs2970471 | 101467854 | 1.0000 | 0.4192 |
| 10 | rs4142734 | 54824741 | 0.5089 | 0.2577 |
| 10 | rs10824875 | 54828992 | 0.5089 | 0.2577 |
| 13 | rs9567867 | 48033401 | 0.8513 | 0.1615 |

| Chr | SNP | Beta | p-value | Variant | Gene |
|---|---|---|---|---|---|
| 7 | rs2970471 | 2.6570 | 3.10e−06 | INTRONIC | CUTL1 |
| 10 | rs4142734 | 2.8220 | 9.45e−06 | INTERGENIC | NA |
| 10 | rs10824875 | 2.8220 | 9.45e−06 | INTERGENIC | NA |
| 13 | rs9567867 | 3.3960 | 6.18e−06 | INTERGENIC | NA |

TABLE 12
Association Results with p < 10⁻⁵ for Endpoint
1 in PGx-CN-HBe-Pos-Interim2, dominant model

| Chr | SNP | BP | HWE(p) | MAF |
|---|---|---|---|---|
| 2 | rs2542943 | 172424963 | 0.7466 | 0.3769 |
| 11 | rs604241 | 133883070 | 0.3933 | 0.3872 |
| 13 | rs9567867 | 48033401 | 0.8513 | 0.1615 |
| 14 | rs4899150 | 65344400 | 0.0986 | 0.4295 |

| Chr | SNP | Beta | p-value | Variant | Gene |
|---|---|---|---|---|---|
| 2 | rs2542943 | 0.2862 | 5.09e−06 | INTERGENIC | NA |
| 11 | rs604241 | 0.2907 | 9.58e−06 | INTERGENIC | NA |
| 13 | rs9567867 | 3.9710 | 3.39e−06 | INTERGENIC | NA |
| 14 | rs4899150 | 0.2996 | 9.96e−06 | INTERGENIC | NA |

TABLE 13
Association Results with p < 10⁻⁵ for Endpoint
1 in PGx-GT-HBe-Pos-Interim2, additive model

| Chr | SNP | BP | HWE(p) | MAF |
|---|---|---|---|---|
| 7 | rs2970471 | 101467854 | 0.6282 | 0.4187 |
| 13 | rs9567867 | 48033401 | 1.0000 | 0.1788 |

| Chr | SNP | Beta | p-value | Variant | Gene |
|---|---|---|---|---|---|
| 7 | rs2970471 | 2.3950 | 5.01e−06 | INTRONIC | CUTL1 |
| 13 | rs9567867 | 3.3230 | 1.86e−06 | INTERGENIC | NA |

TABLE 14
Association Results with p < 10⁻⁵ for Endpoint
1 in PGx-GT-HBe-Pos-Interim2, dominant model

| Chr | SNP | BP | HWE(p) | MAF |
|---|---|---|---|---|
| 2 | rs2542943 | 172424963 | 0.0044 | 0.4721 |
| 5 | rs508636 | 80311835 | 0.1432 | 0.2248 |
| 13 | rs9567867 | 48033401 | 1.0000 | 0.1788 |
| 14 | rs4899150 | 65344400 | 2.69e−10 | 0.3909 |
| 21 | rs12626242 | 34111588 | 1.0000 | 0.0759 |

| Chr | SNP | Beta | p-value | Variant | Gene |
|---|---|---|---|---|---|
| 2 | rs2542943 | 0.2931 | 1.85e−06 | INTERGENIC | NA |
| 5 | rs508636 | 3.1830 | 9.91e−06 | INTRONIC | RASGRF2 |
| 13 | rs9567867 | 3.9500 | 7.61e−07 | INTERGENIC | NA |
| 14 | rs4899150 | 0.2958 | 8.41e−06 | INTERGENIC | NA |
| 21 | rs12626242 | 3.8680 | 8.38e−06 | INTRONIC | C21orf66 |

### Results for Endpoint 2

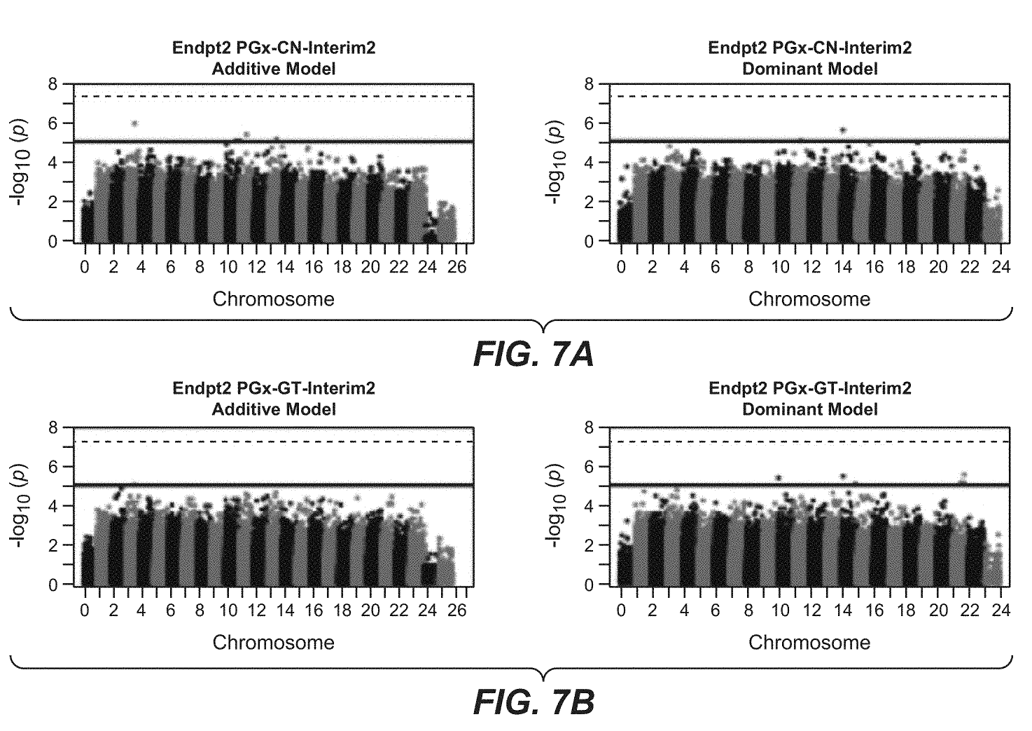

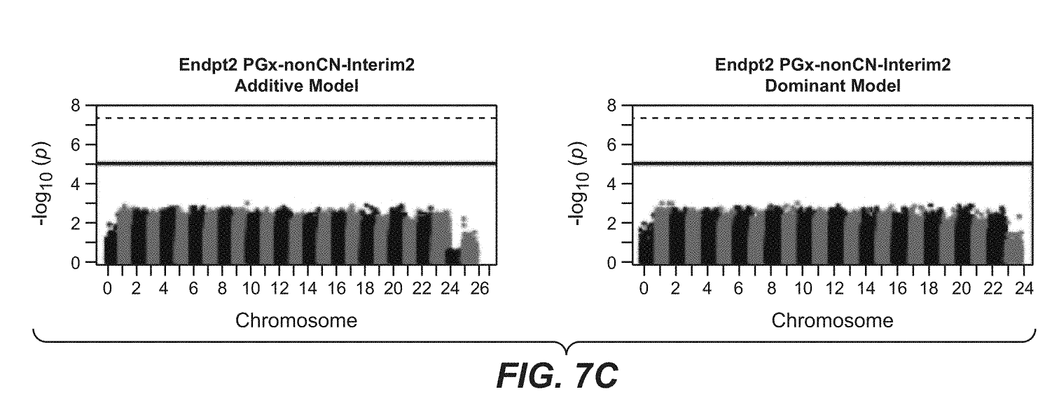

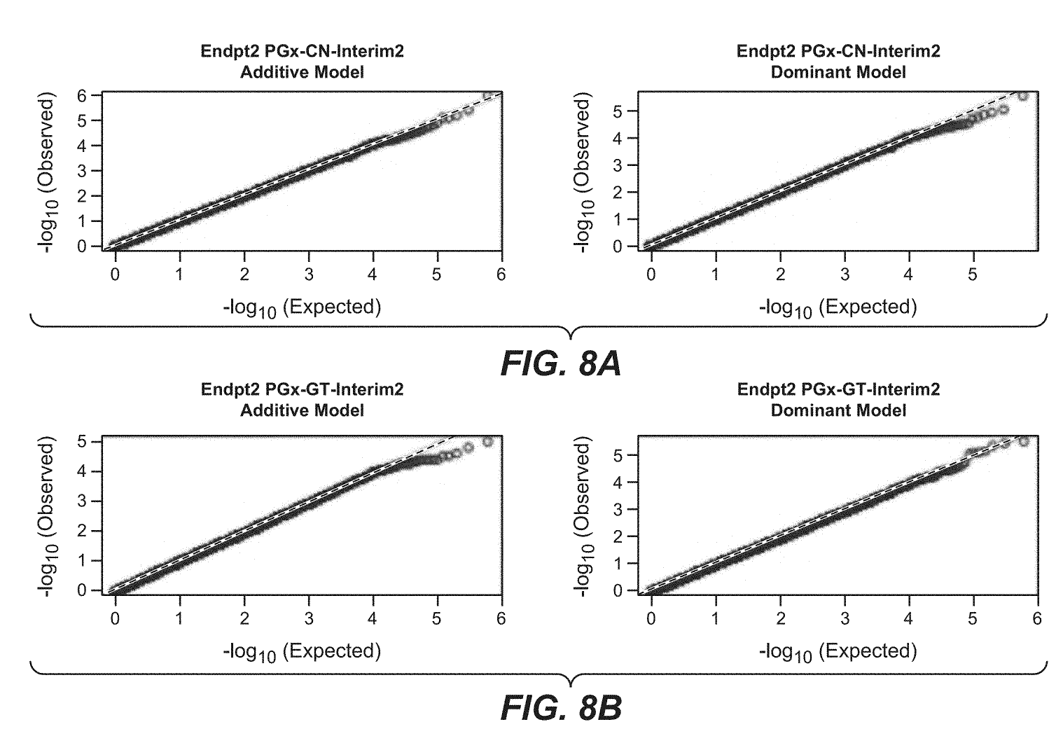

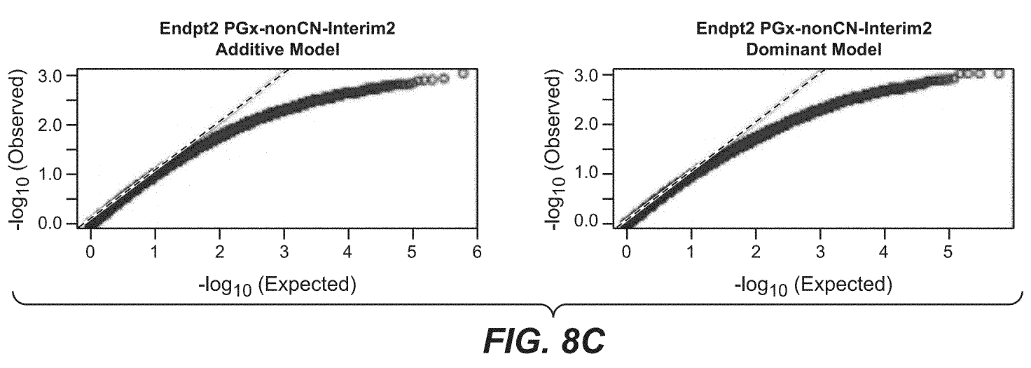

FIGS. 7 and 8 show the Manhattan Plots and QQ plots respectively, for Endpoint 2. Details of markers with p<10⁻⁵ are given in Tables 15-18. No marker had p<10⁻⁵ in PGx-nonCN-HBe-Pos-Interim2, under either mode of inheritance however, there were only 11 responders in this group. The QQ-plots were seen to curve downwards and the Manhattan plots were depressed.

TABLE 15
Association Results with p < 10⁻⁵ for Endpoint
2 in PGx-CN-HBe-Pos-Interim2, additive model

| Chr | SNP | BP | HWE(p) | MAF |
|---|---|---|---|---|
| 3 | rs7633796 | 62758410 | 0.1011 | 0.1897 |
| 10 | rs4142734 | 54824741 | 0.5089 | 0.2577 |
| 10 | rs10824875 | 54828992 | 0.5089 | 0.2577 |
| 11 | rs7947950 | 117312089 | 0.4865 | 0.1256 |
| 13 | rs12584550 | 111769770 | 0.2126 | 0.3372 |

| Chr | SNP | Beta | p-value | Variant | Gene |
|---|---|---|---|---|---|
| 3 | rs7633796 | 3.4130 | 1.17e−06 | INTRONIC | CADPS |
| 10 | rs4142734 | 3.1980 | 8.96e−06 | INTERGENIC | NA |
| 10 | rs10824875 | 3.1980 | 8.96e−06 | INTERGENIC | NA |
| 11 | rs7947950 | 4.2420 | 4.43e−06 | INTRONIC | DSCAML1 |
| 13 | rs12584550 | 2.9350 | 7.14e−06 | INTRONIC | ARHGEF7 |

TABLE 16
Association Results with p < 10⁻⁵ for Endpoint
2 in PGx-CN-HBe-Pos-Interim2, dominant model

| Chr | SNP | BP | HWE(p) | MAF |
|---|---|---|---|---|
| 11 | rs7947950 | 117312089 | 0.4865 | 0.1256 |
| 14 | rs4899150 | 65344400 | 0.0986 | 0.4295 |

| Chr | SNP | Beta | p-value | Variant | Gene |
|---|---|---|---|---|---|
| 11 | rs7947950 | 4.4610 | 9.28e−06 | INTRONIC | DSCAML1 |
| 14 | rs4899150 | 0.2208 | 2.81e−06 | INTERGENIC | NA |

TABLE 17
Association Results with p < 10⁻⁵ for Endpoint
2 in PGx-GT-HBe-Pos-Interim2, additive model

| Chr | SNP | BP | HWE(p) | MAF |
|---|---|---|---|---|
| 3 | rs7633796 | 62758410 | 0.0081 | 0.2786 |

| Chr | SNP | Beta | p-value | Variant | Gene |
|---|---|---|---|---|---|
| 3 | rs7633796 | 2.7920 | 8.40e−06 | INTRONIC | CADPS |

TABLE 18
Association Results with p < 10⁻⁵ for Endpoint
2 in PGx-GT-HBe-Pos-Interim2, dominant model

| Chr | SNP | BP | HWE(p) | MAF |
|---|---|---|---|---|
| 10 | rs10765101 | 129246566 | 0.4655 | 0.1602 |
| 14 | rs4899150 | 65344400 | 2.69e−10 | 0.3909 |
| 14 | rs12435908 | 66121468 | 5.65e−07 | 0.3104 |
| 21 | rs845023 | 34000493 | 1.0000 | 0.0760 |
| 21 | rs12627478 | 34114123 | 1.0000 | 0.0759 |
| 21 | rs12626242 | 34111588 | 1.0000 | 0.0759 |
| 21 | rs10470165 | 34020653 | 1.0000 | 0.0760 |

| Chr | SNP | Beta | p-value | Variant | Gene |
|---|---|---|---|---|---|
| 10 | rs10765101 | 4.2420 | 3.97e−06 | INTRONIC | DOCK1 |
| 14 | rs4899150 | 0.2231 | 3.28e−06 | INTERGENIC | NA |
| 14 | rs12435908 | 5.6830 | 8.29e−06 | INTRONIC | FUT8 |
| 21 | rs845023 | 4.3210 | 7.31e−06 | DOWNSTREAM | NA |
| 21 | rs12627478 | 4.3720 | 6.38e−06 | INTRONIC | C21orf66 |
| 21 | rs12626242 | 4.5990 | 2.73e−06 | INTRONIC | C21orf66 |
| 21 | rs10470165 | 4.3290 | 7.14e−06 | INTRONIC | SYNJ1 |

### Results for Endpoint 3

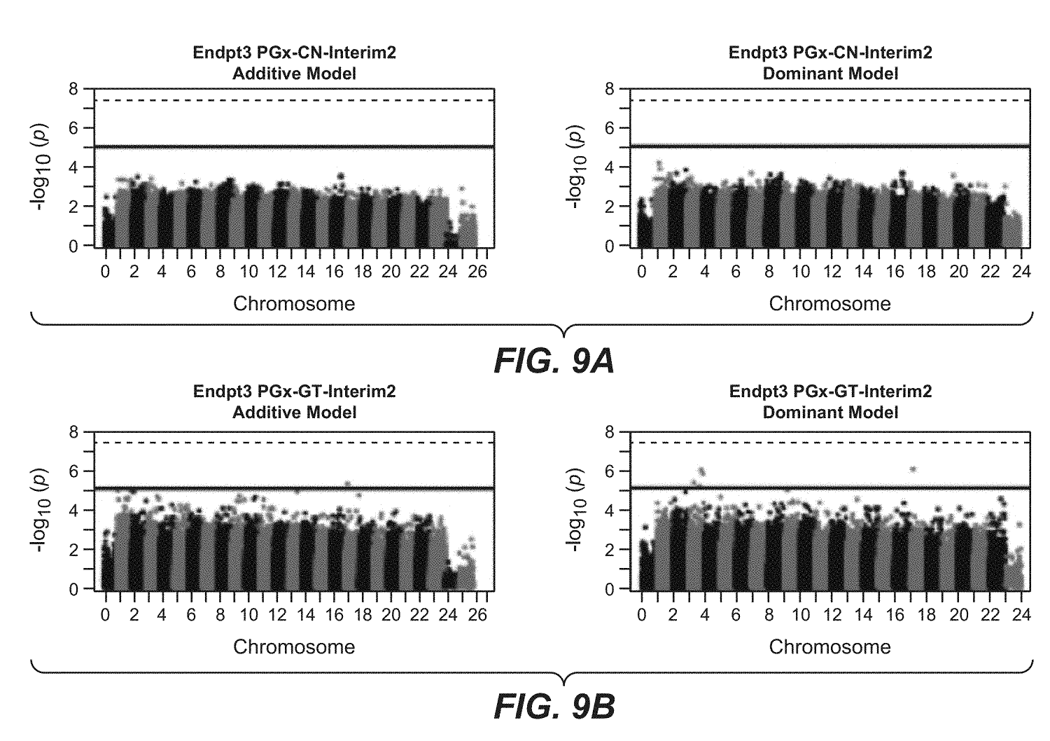

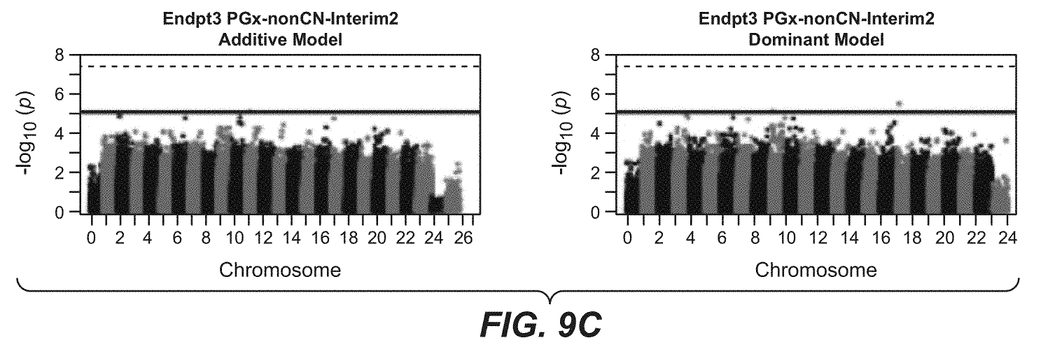

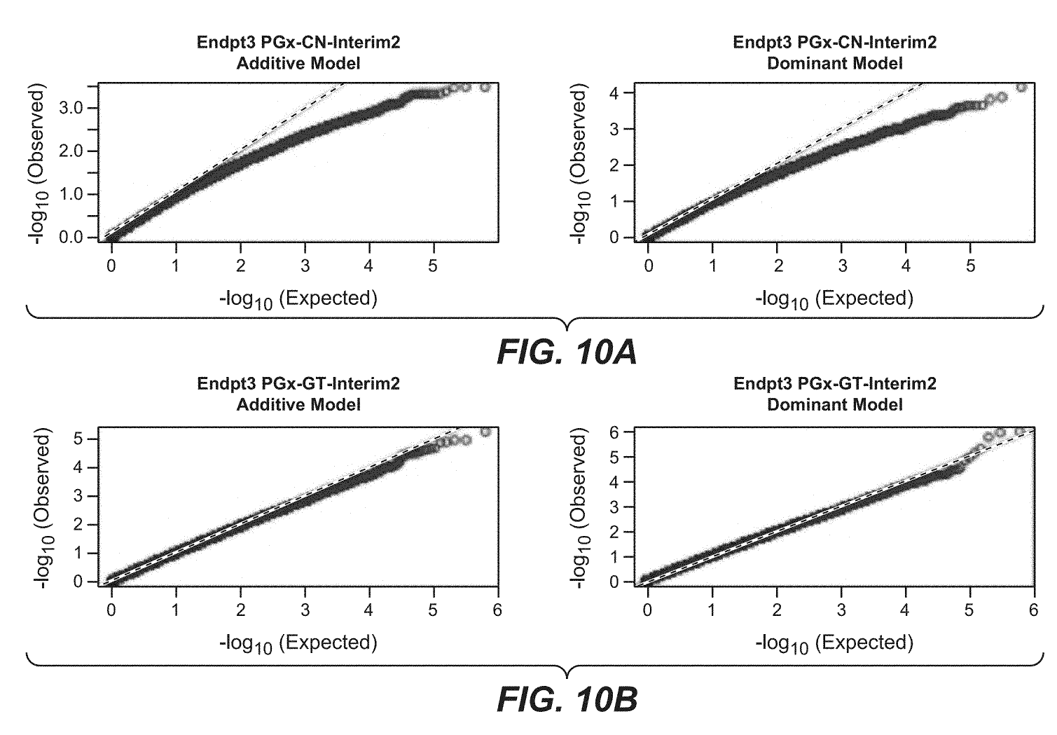

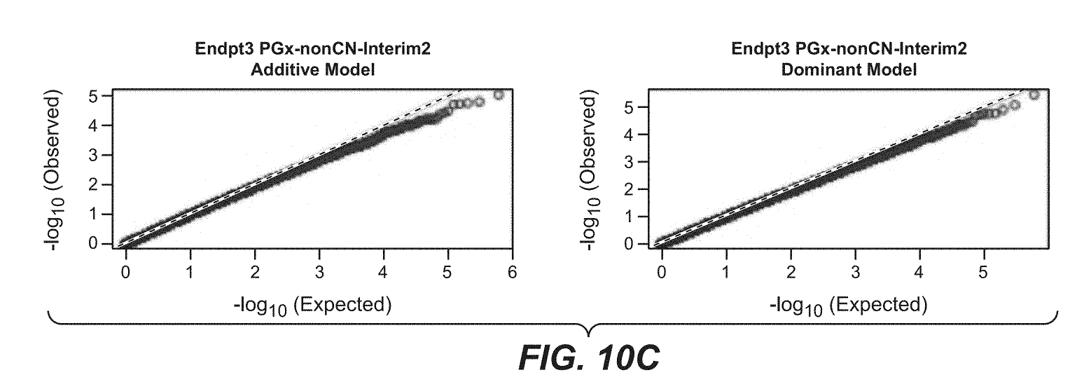

FIGS. 9 and 10 show the Manhattan Plots and QQ plots respectively, for Endpoint 3. Details of markers with p<10⁻⁵ are given in Tables 19-22. No marker had p<10⁻⁵ in PGx-CN-HBe-Neg-Interim2, under either mode of inheritance however, there were only 16 responders in this group. The QQ-plots were seen to curve downwards and the Manhattan plots were depressed.

TABLE 19
Association Results with p < 10⁻⁵ for Endpoint
3 in PGx-GT-Hbe-Neg-Interim2, additive model

| Chr | SNP | BP | HWE(p) | MAF |
|---|---|---|---|---|
| 17 | rs1867475 | 76507025 | 0.7401 | 0.3870 |

| Chr | SNP | Beta | p-value | Variant | Gene |
|---|---|---|---|---|---|
| 17 | rs1867475 | 0.3168 | 5.46e−06 | INTERGENIC | NA |

TABLE 20
Association Results with p < 10⁻⁵ for Endpoint
3 in PGx-GT- Hbe-Neg-Interim2, dominant model

| Chr | SNP | BP | HWE(p) | MAF |
|---|---|---|---|---|
| 3 | rs9856296 | 14155453 | 0.8630 | 0.3498 |
| 3 | rs6776709 | 13978803 | 1.0000 | 0.3787 |
| 3 | rs4684175 | 14048394 | 0.8653 | 0.3651 |
| 3 | rs4685060 | 14042534 | 0.7342 | 0.3646 |
| 17 | rs1867475 | 76507025 | 0.7401 | 0.3870 |

| Chr | SNP | Beta | p-value | Variant | Gene |
|---|---|---|---|---|---|
| 3 | rs9856296 | 4.4270 | 5.26e−06 | INTRONIC | CHCHD4 |
| 3 | rs6776709 | 4.0870 | 8.24e−06 | INTERGENIC | NA |
| 3 | rs4684175 | 4.7410 | 1.23e−06 | INTERGENIC | NA |
| 3 | rs4685060 | 4.6310 | 1.87e−06 | INTERGENIC | NA |
| 17 | rs1867475 | 0.1408 | 1.10e−06 | INTERGENIC | NA |

TABLE 21
Association Results with p < 10⁻⁵ for Endpoint
3 in PGx-nonCN- Hbe-Neg-Interim2, additive model

| Chr | SNP | BP | HWE(p) | MAF |
|---|---|---|---|---|
| 11 | rs4539348 | 71242461 | 0.6187 | 0.2480 |

| Chr | SNP | Beta | p-value | Variant | Gene |
|---|---|---|---|---|---|
| 11 | rs4539348 | 4.4530 | 8.89e−06 | INTRONIC | KRTAP5-7; |
| | | | | | KRTAP5-8 |

TABLE 22
Association Results with p < 10⁻⁵ for Endpoint
3 in PGx-nonCN- Hbe-Neg-Interim2, dominant model

| Chr | SNP | BP | HWE(p) | MAF |
|---|---|---|---|---|
| 9 | rs13297144 | 6788085 | 0.2541 | 0.2598 |
| 17 | rs1867475 | 76507025 | 0.6155 | 0.4668 |

| Chr | SNP | Beta | p-value | Variant | Gene |
|---|---|---|---|---|---|
| 9 | rs13297144 | 0.1848 | 8.41e−06 | INTRONIC | JMJD2C |
| 17 | rs1867475 | 0.0844 | 3.48e−06 | INTERGENIC | NA |

### Results for Endpoint 4

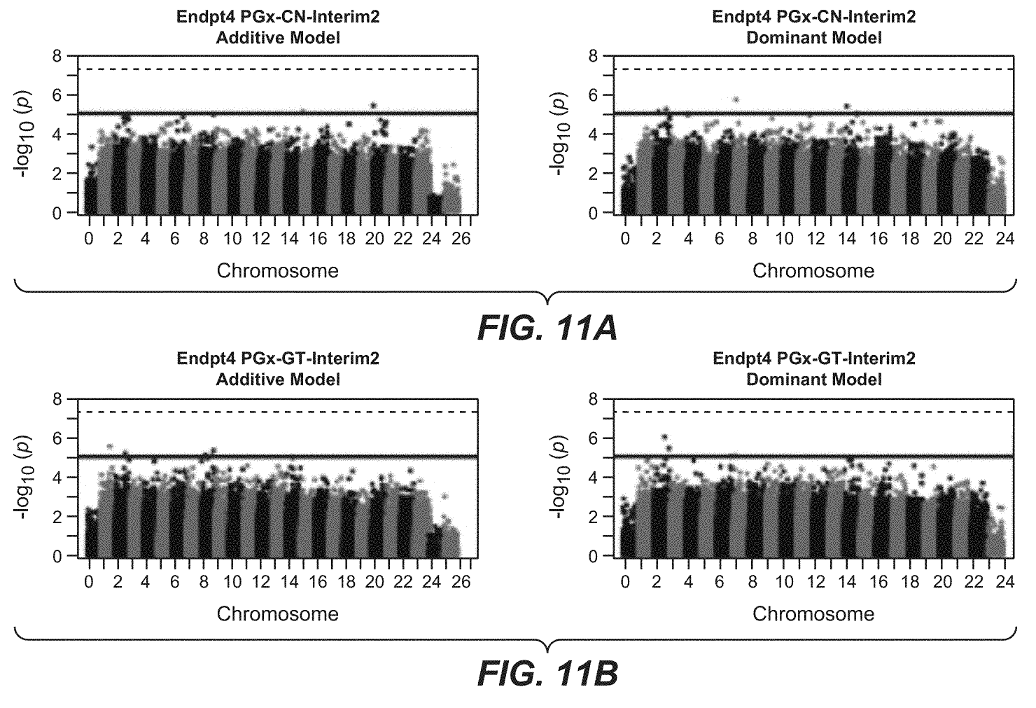

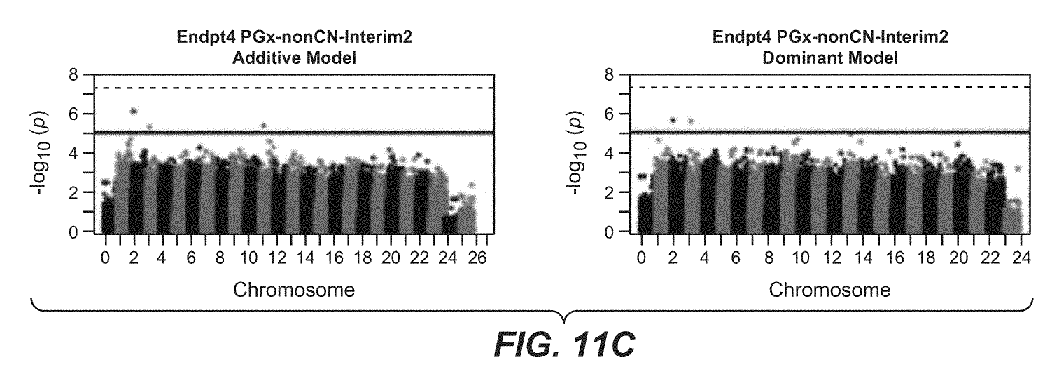

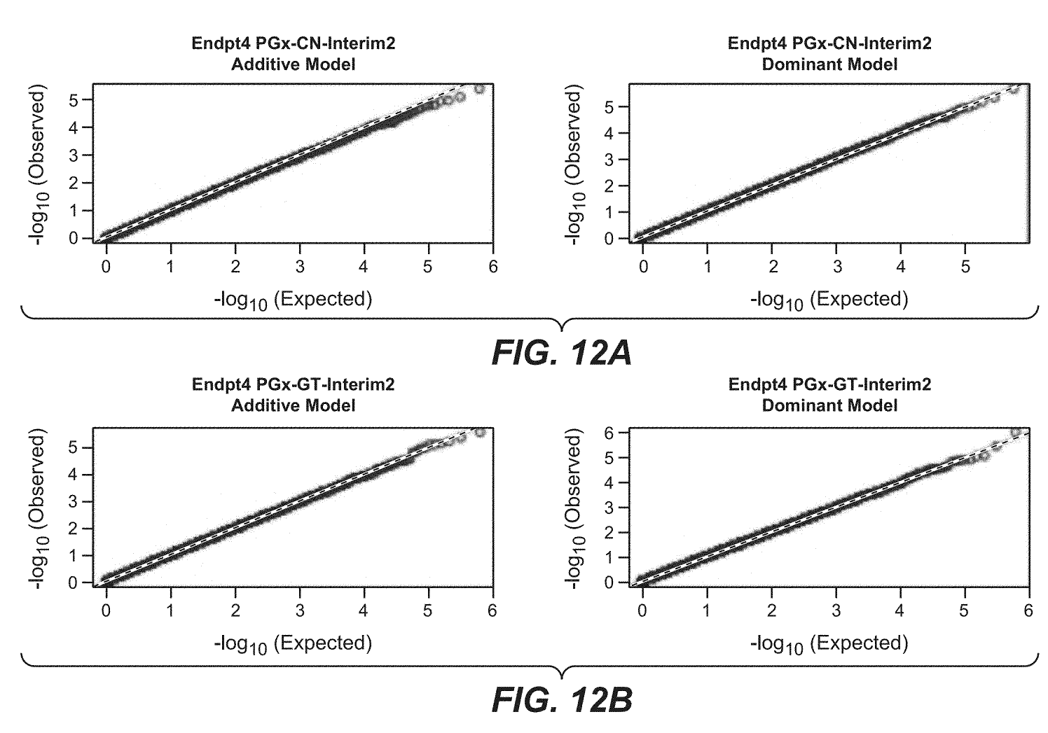

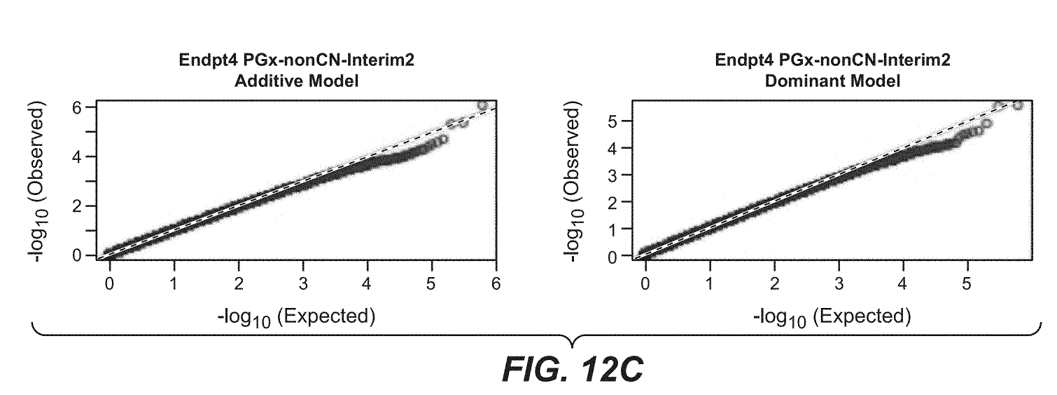

FIGS. 11 and 12 show the Manhattan Plots and QQ plots respectively, for Endpoint 4. Details of markers with p<10⁻⁵ are given in Tables 23-28.

TABLE 23
Association Results with p < 10⁻⁵ for Endpoint 4 in
PGx-CN-Interim2, additive model

| Chr | SNP | BP | HWE(p) | MAF |
|---|---|---|---|---|
| 15 | rs11072478 | 74615507 | 0.3785 | 0.3551 |
| 20 | rs260010 | 57764298 | 0.9140 | 0.3756 |

| Chr | SNP | Beta | p-value | Variant | Gene |
|---|---|---|---|---|---|
| 15 | rs11072478 | 2.3750 | 7.69e−06 | INTRONIC | CCDC33 |
| 20 | rs260010 | 0.3921 | 3.87e−06 | UPSTREAM | NA |

TABLE 24
Association Results with p < 10⁻⁵ for Endpoint 4 in
PGx-CN-Interim2, dominant model

| Chr | SNP | BP | HWE(p) | MAF |
|---|---|---|---|---|
| 2 | rs13395925 | 7893850 | 0.3211 | 0.2859 |
| 2 | rs2542943 | 172424963 | 0.7466 | 0.3769 |
| 7 | rs845562 | 55254805 | 0.6640 | 0.3705 |
| 14 | rs4899150 | 65344400 | 0.0986 | 0.4295 |

| Chr | SNP | Beta | p-value | Variant | Gene |
|---|---|---|---|---|---|
| 2 | rs13395925 | 0.3121 | 7.87e−06 | INTERGENIC | NA |
| 2 | rs2542943 | 0.3149 | 6.07e−06 | INTERGENIC | NA |
| 7 | rs845562 | 3.8440 | 1.91e−06 | INTRONIC | EGFR |
| 14 | rs4899150 | 0.2940 | 4.15e−06 | INTERGENIC | NA |

TABLE 25
Association Results with p < 10⁻⁵ for Endpoint 4 in PGx-GT-Interim2, additive model

| Chr | SNP | BP | HWE(p) | MAF |
|---|---|---|---|---|
| 1 | rs12568559 | 116223259 | 1.0000 | 0.3297 |
| 2 | rs10193128 | 233987722 | 0.5085 | 0.3909 |
| NA | exm680251 | NA | 0.0226 | 0.4938 |
| 8 | rs4840946 | 8231793 | 0.0335 | 0.4961 |
| 8 | rs4840947 | 8232143 | 0.0145 | 0.4837 |
| NA | exm680207 | NA | 0.0277 | 0.4961 |

| Chr | SNP | Beta | p-value | Variant | Gene |
|---|---|---|---|---|---|
| 1 | rs12568559 | 0.4741 | 2.69e−06 | INTRONIC | VANGL1 |
| 2 | rs10193128 | 0.5201 | 6.18e−06 | INTRONIC | ENSG00000168918 |
| NA | exm680251 | 1.8990 | 8.12e−06 | NA | NA |
| 8 | rs4840946 | 1.8980 | 7.53e−06 | INTRONIC | ENSG00000182319 |
| 8 | rs4840947 | 1.8830 | 8.19e−06 | INTRONIC | ENSG00000182319 |
| NA | exm680207 | 1.9370 | 4.43e−06 | NA | NA |

TABLE 26
Association Results with p < 10⁻⁵ for Endpoint 4 in PGx-GT-Interim2, dominant model

| Chr | SNP | BP | HWE(p) | MAF |
|---|---|---|---|---|
| 2 | rs10193128 | 233987722 | 0.5085 | 0.3909 |
| 2 | rs9288685 | 233987114 | 0.6287 | 0.4218 |
| 6 | rs9322467 | 154771277 | 0.0324 | 0.1796 |
| 7 | rs845562 | 55254805 | 0.0329 | 0.2833 |

| Chr | SNP | Beta | p-value | Variant | Gene |
|---|---|---|---|---|---|
| 2 | rs10193128 | 0.3693 | 9.53e−07 | INTRONIC | ENSG00000168918 |
| 2 | rs9288685 | 0.3791 | 3.60e−06 | INTRONIC | ENSG00000168918 |
| 6 | rs9322467 | 2.5710 | 9.31e−06 | SYNONYMOUS | CNKSR3 |
| | | | | CODING | |
| 7 | rs845562 | 2.6040 | 8.61e−06 | INTRONIC | EGFR |

TABLE 27
Association Results with p < 10⁻⁵ for Endpoint 4 in
PGx-nonCN-Interim2, additive model

| Chr | SNP | BP | HWE(p) | MAF |
|---|---|---|---|---|
| 2 | rs6733352 | 209472769 | 0.0138 | 0.4118 |
| 3 | rs7633147 | 55161838 | 1.0000 | 0.2246 |
| 11 | rs4539348 | 71242461 | 0.6187 | 0.2480 |

| Chr | SNP | Beta | p-value | Variant | Gene |
|---|---|---|---|---|---|
| 2 | rs6733352 | 0.2659 | 7.73e−07 | INTERGENIC | NA |
| 3 | rs7633147 | 0.2355 | 4.76e−06 | INTERGENIC | NA |
| 11 | rs4539348 | 4.4900 | 4.08e−06 | INTRONIC | KRTAP5-7; |
| | | | | | KRTAP5-8 |

TABLE 28
Association Results with p < 10⁻⁵ for Endpoint 4 in
PGx-nonCN-Interim2, dominant model

| Chr | SNP | BP | HWE(p) | MAF |
|---|---|---|---|---|
| 2 | rs6733352 | 209472769 | 0.0138 | 0.4118 |
| 3 | rs7633147 | 55161838 | 1.0000 | 0.2246 |

| Chr | SNP | Beta | p-value | Variant | Gene |
|---|---|---|---|---|---|
| 2 | rs6733352 | 0.1382 | 2.40e−06 | INTERGENIC | NA |
| 3 | rs7633147 | 0.1857 | 2.71e−06 | INTERGENIC | NA |

### Results for Endpoint 5

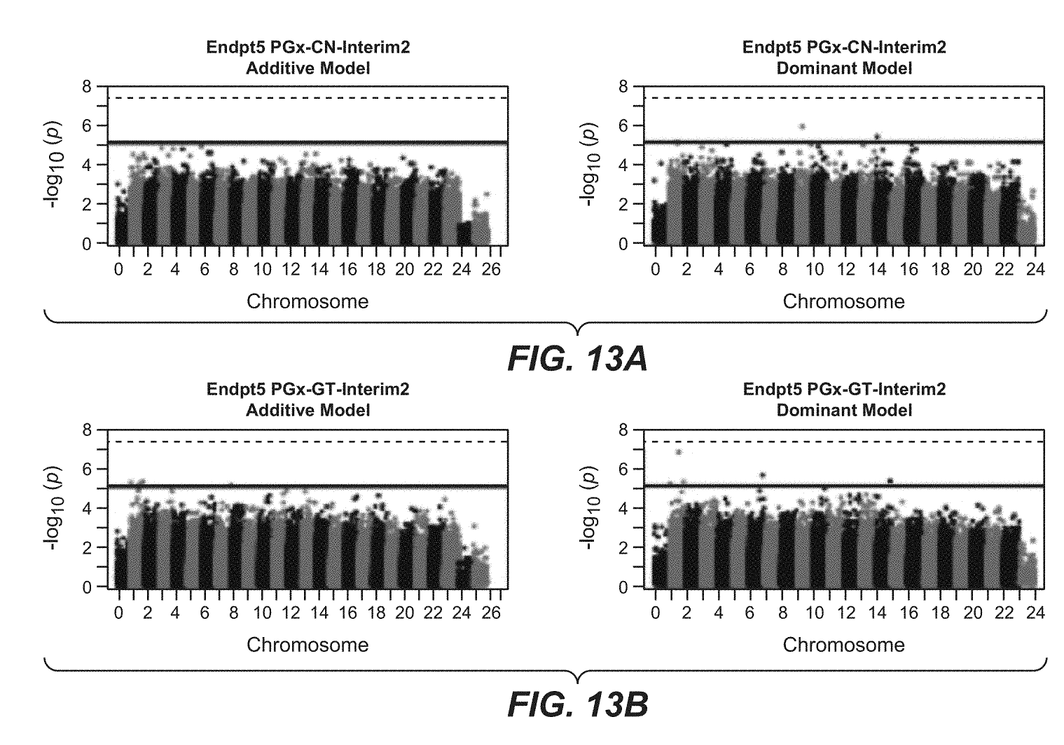

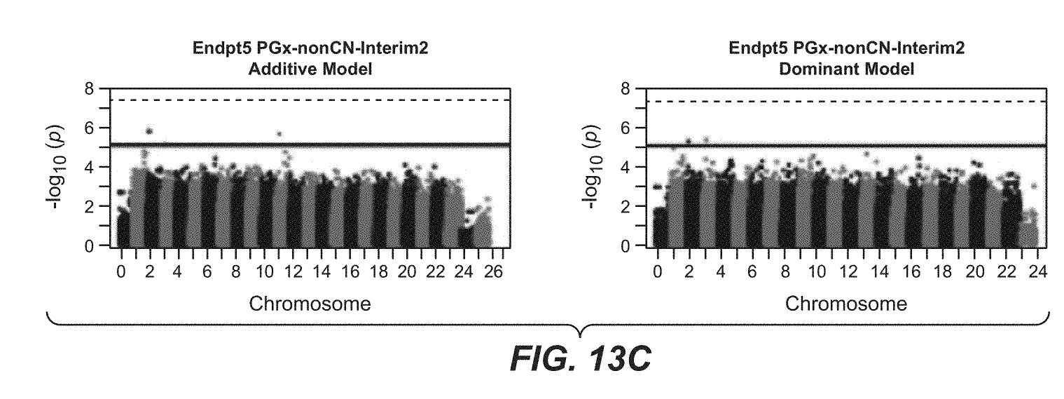

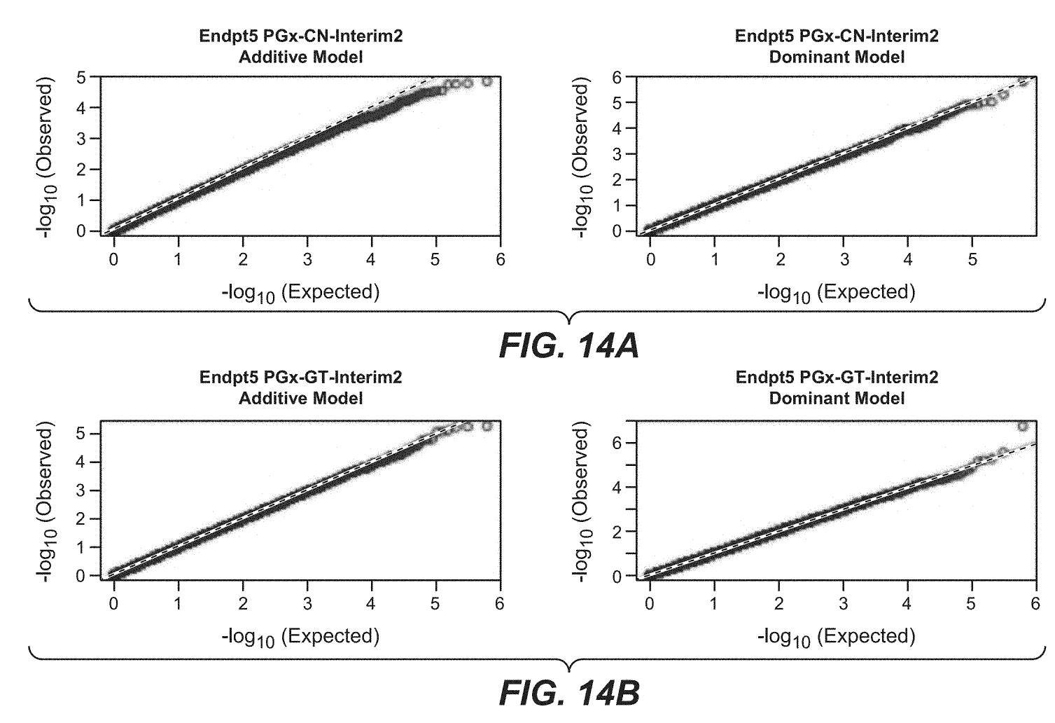

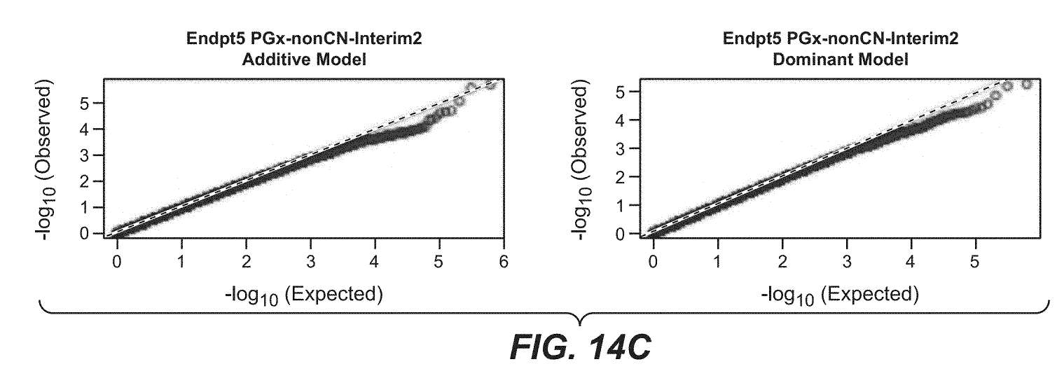

FIGS. 13 and 14 show the Manhattan Plots and QQ plots respectively, for Endpoint 5. Details of markers with p<10⁻⁵ are given in Tables 29-33.

TABLE 29
Association Results with p < 10⁻⁵ for Endpoint 5 in
PGx-CN-Interim2, dominant model

| Chr | SNP | BP | HWE(p) | MAF |
|---|---|---|---|---|
| 9 | rs943172 | 7403305 | 0.6864 | 0.4833 |
| 14 | rs4899150 | 65344400 | 0.0986 | 0.4295 |

| Chr | SNP | Beta | p-value | Variant | Gene |
|---|---|---|---|---|---|
| 9 | rs943172 | 0.2428 | 1.54e−06 | INTERGENIC | NA |
| 14 | rs4899150 | 0.2515 | 5.05e−06 | INTERGENIC | NA |

TABLE 30
Association Results with p < 10⁻⁵ for Endpoint 5 in PGx-GT-Interim2, additive model

| Chr | SNP | BP | HWE(p) | MAF |
|---|---|---|---|---|
| 1 | rs12568559 | 116223259 | 1.0000 | 0.3297 |
| 1 | rs4348723 | 116239633 | 0.2267 | 0.3034 |
| 1 | rs6428677 | 116240026 | 0.1912 | 0.3019 |
| 1 | rs4654771 | 22158363 | 0.0332 | 0.4745 |
| 1 | rs2500499 | 244271051 | 0.1647 | 0.1711 |
| 8 | rs4840939 | 8215349 | 0.3844 | 0.4659 |

| Chr | SNP | Beta | p-value | Variant | Gene |
|---|---|---|---|---|---|
| 1 | rs12568559 | 0.4597 | 7.41e−06 | INTRONIC | VANGL1 |
| 1 | rs4348723 | 0.4420 | 5.07e−06 | 3PRIME_UTR | VANGL1 |
| 1 | rs6428677 | 0.4447 | 5.84e−06 | 3PRIME_UTR | VANGL1 |
| 1 | rs4654771 | 0.5019 | 8.30e−06 | INTRONIC | HSPG2 |
| 1 | rs2500499 | 2.3800 | 5.44e−06 | INTERGENIC | NA |
| 8 | rs4840939 | 0.5054 | 8.19e−06 | INTRONIC | ENSG00000182319 |

TABLE 31
Association Results with p < 10⁻⁵ for Endpoint 5 in
PGx-GT-Interim2, dominant model

| Chr | SNP | BP | HWE(p) | MAF |
|---|---|---|---|---|
| 1 | rs4348723 | 116239633 | 0.2267 | 0.3034 |
| 1 | rs6428677 | 116240026 | 0.1912 | 0.3019 |
| 1 | rs4654771 | 22158363 | 0.0332 | 0.4745 |
| 6 | rs9322467 | 154771277 | 0.0324 | 0.1796 |
| 14 | rs12435908 | 66121468 | 5.65e−07 | 0.3104 |

| Chr | SNP | Beta | p-value | Variant | Gene |
|---|---|---|---|---|---|
| 1 | rs4348723 | 0.3687 | 6.06e−06 | 3PRIME_UTR | VANGL1 |
| 1 | rs6428677 | 0.3720 | 7.24e−06 | 3PRIME_UTR | VANGL1 |
| 1 | rs4654771 | 0.2781 | 1.81e−07 | INTRONIC | HSPG2 |
| 6 | rs9322467 | 2.8850 | 2.74e−06 | SYNONYMOUS | CNKSR3 |
| | | | | CODING | |
| 14 | rs12435908 | 3.3790 | 5.40e−06 | INTRONIC | FUT8 |

TABLE 32
Association Results with p < 10⁻⁵ for Endpoint
5 in PGx-nonCN-Interim2, additive model

| Chr | SNP | BP | HWE(p) | MAF |
|---|---|---|---|---|
| 2 | rs6733352 | 209472769 | 0.0138 | 0.4118 |
| 3 | rs7633147 | 55161838 | 1.0000 | 0.2246 |
| 11 | rs4539348 | 71242461 | 0.6187 | 0.2480 |

| Chr | SNP | Beta | p-value | Variant | Gene |
|---|---|---|---|---|---|
| 2 | rs6733352 | 0.2849 | 1.79e−06 | INTERGENIC | NA |
| 3 | rs7633147 | 0.2460 | 8.05e−06 | INTERGENIC | NA |
| 11 | rs4539348 | 4.6940 | 2.46e−06 | INTRONIC | KRTAP5- |
| | | | | | 7; KRTAP5-8 |

TABLE 33
Association Results with p < 10⁻⁵ for Endpoint 5 in
PGx-nonCN-Interim2, dominant model

| Chr | SNP | BP | HWE(p) | MAF |
|---|---|---|---|---|
| 2 | rs6733352 | 209472769 | 0.0138 | 0.4118 |
| 3 | rs7633147 | 55161838 | 1.0000 | 0.2246 |

| Chr | SNP | Beta | p-value | Variant | Gene |
|---|---|---|---|---|---|
| 2 | rs6733352 | 0.1577 | 5.91e−06 | INTERGENIC | NA |
| 3 | rs7633147 | 0.1958 | 4.93e−06 | INTERGENIC | NA |

### Results for Endpoint 6

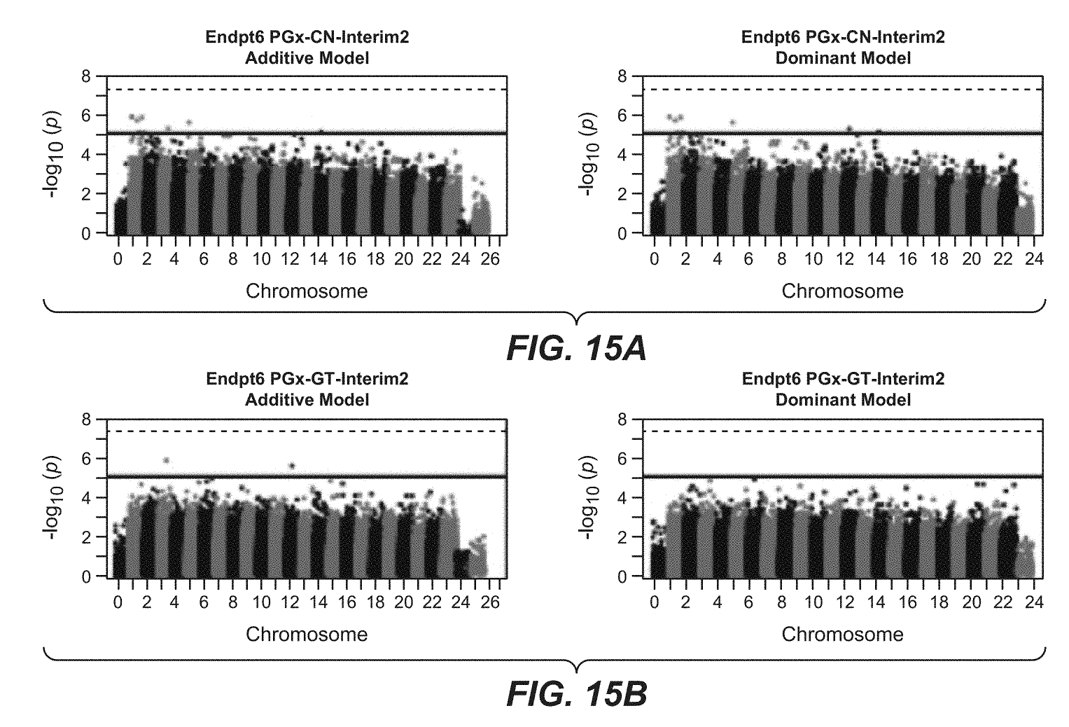

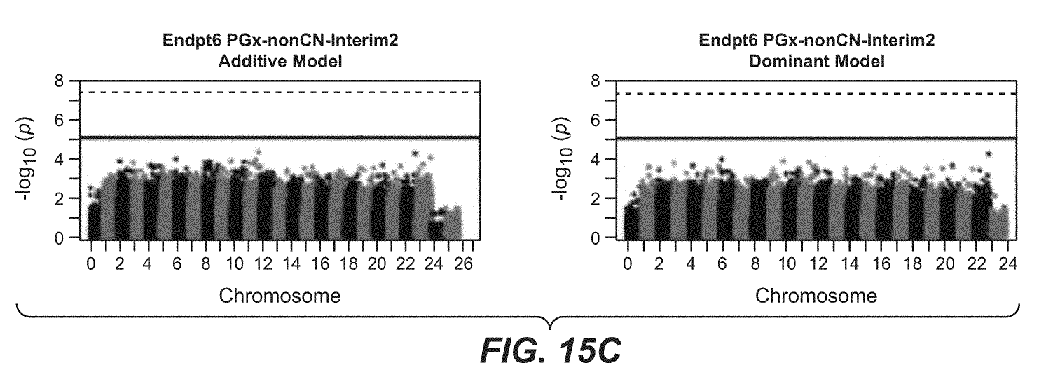

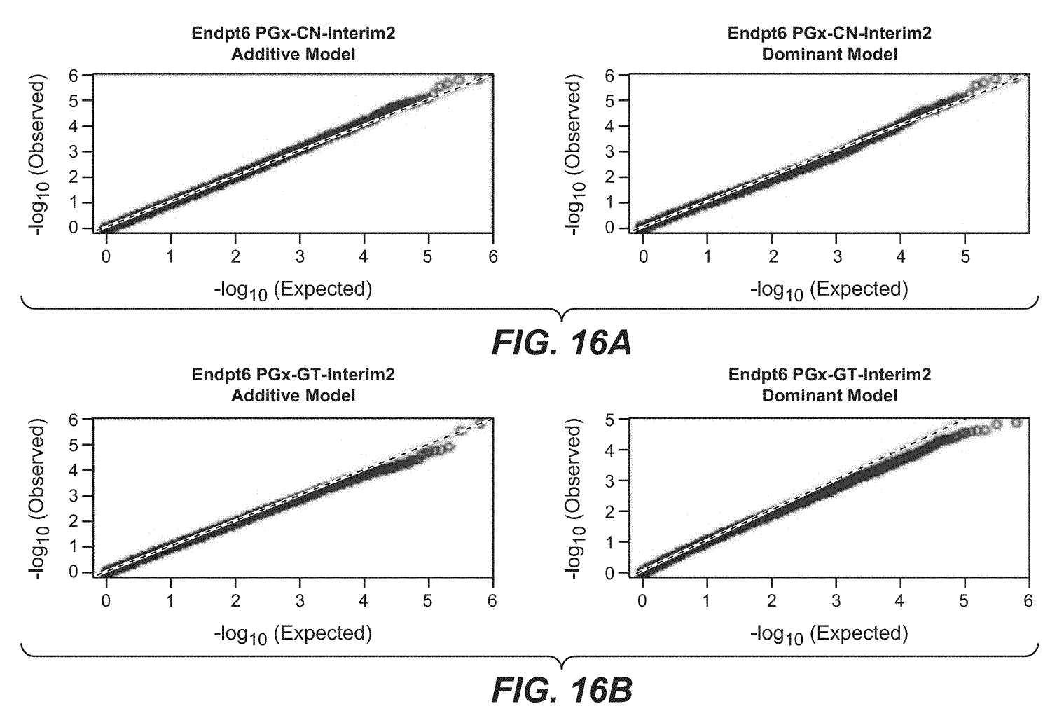

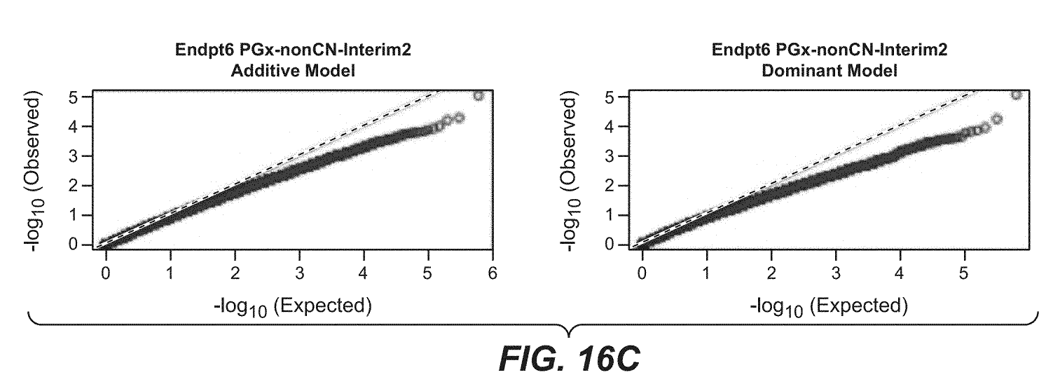

FIGS. 15 and 16 show the Manhattan Plots and QQ plots respectively, for Endpoint 6. Details of markers with p<10⁻⁵ are given in Tables 34-37.

TABLE 34
Association Results with p < 10⁻⁵ for Endpoint 6 in
PGx-CN-Interim2, additive model

| Chr | SNP | BP | HWE(p) | MAF |
|---|---|---|---|---|
| 1 | rs10494323 | 159374604 | 1.0000 | 0.0205 |
| NA | exm-rs4133289 | NA | 1.0000 | 0.0218 |
| NA | exm-rs10489849 | NA | 1.0000 | 0.0167 |
| NA | exm114297 | NA | 1.0000 | 0.0205 |
| 1 | rs10489849 | 159196765 | 1.0000 | 0.0167 |
| 3 | rs17403692 | 65104083 | 0.1494 | 0.0961 |
| 5 | rs2662605 | 17999554 | 1.0000 | 0.0295 |
| 14 | rs17094430 | 97244170 | 1.0000 | 0.0180 |

| Chr | SNP | Beta | p-value | Variant | Gene |
|---|---|---|---|---|---|
| 1 | rs10494323 | 25.9100 | 1.47e−06 | INTRONIC | OR10J1 |
| NA | exm-rs4133289 | 23.3500 | 2.20e−06 | NA | NA |
| NA | exm-rs10489849 | 22.0200 | 9.15e−06 | NA | NA |
| NA | exm114297 | 25.8000 | 1.57e−06 | NA | NA |
| 1 | rs10489849 | 22.0200 | 9.15e−06 | INTERGENIC | NA |
| 3 | rs17403692 | 8.7050 | 5.71e−06 | INTERGENIC | NA |
| 5 | rs2662605 | 24.7700 | 2.84e−06 | INTERGENIC | NA |
| 14 | rs17094430 | 26.7600 | 8.62e−06 | INTERGENIC | NA |

TABLE 35
Association Results with p < 10⁻⁵ for Endpoint 6 in
PGx-CN-Interim2, dominant model

| Chr | SNP | BP | HWE(p) | MAF |
|---|---|---|---|---|
| 1 | rs10494323 | 159374604 | 1.0000 | 0.0205 |
| NA | exm-rs4133289 | NA | 1.0000 | 0.0218 |
| NA | exm-rs10489849 | NA | 1.0000 | 0.0167 |
| NA | exm114297 | NA | 1.0000 | 0.0205 |
| 1 | rs10489849 | 159196765 | 1.0000 | 0.0167 |
| 5 | rs2662605 | 17999554 | 1.0000 | 0.0295 |
| 12 | rs11110982 | 102118934 | 0.2016 | 0.0244 |
| 14 | rs17094430 | 97244170 | 1.0000 | 0.0180 |

| Chr | SNP | Beta | p-value | Variant | Gene |
|---|---|---|---|---|---|
| 1 | rs10494323 | 25.9100 | 1.47e−06 | INTRONIC | OR10J1 |
| NA | exm-rs4133289 | 23.3500 | 2.20e−06 | NA | NA |
| NA | exm-rs10489849 | 22.0200 | 9.15e−06 | NA | NA |
| NA | exm114297 | 25.8000 | 1.57e−06 | NA | NA |
| 1 | rs10489849 | 22.0200 | 9.15e−06 | INTERGENIC | NA |
| 5 | rs2662605 | 24.7700 | 2.84e−06 | INTERGENIC | NA |
| 12 | rs11110982 | 19.0800 | 6.07e−06 | INTRONIC | CHPT1 |
| 14 | rs17094430 | 26.7600 | 8.62e−06 | INTERGENIC | NA |

TABLE 36
Association Results with p < 10⁻⁵ for Endpoint 6 in
PGx-GT-Interim2, additive model

| Chr | SNP | BP | HWE(p) | MAF |
|---|---|---|---|---|
| 3 | rs17403692 | 65104083 | 8.71e−06 | 0.2183 |
| 12 | rs11177673 | 69832861 | 0.6131 | 0.1927 |

| Chr | SNP | Beta | p-value | Variant | Gene |
|---|---|---|---|---|---|
| 3 | rs17403692 | 5.5880 | 1.54e−06 | INTERGENIC | NA |
| 12 | rs11177673 | 5.2320 | 2.94e−06 | INTERGENIC | NA |

TABLE 37
Association Results with p < 10⁻⁵ for Endpoint 6 in
PGx-nonCN-Interim2, additive model

| Chr | SNP | BP | HWE(p) | MAF |
|---|---|---|---|---|
| 19 | rs736452 | 33145645 | 1.0000 | 0.0352 |

| Chr | SNP | Beta | p-value | Variant | Gene |
|---|---|---|---|---|---|
| 19 | rs736452 | 47.9600 | 9.31e−06 | INTRONIC | ANKRD27 |

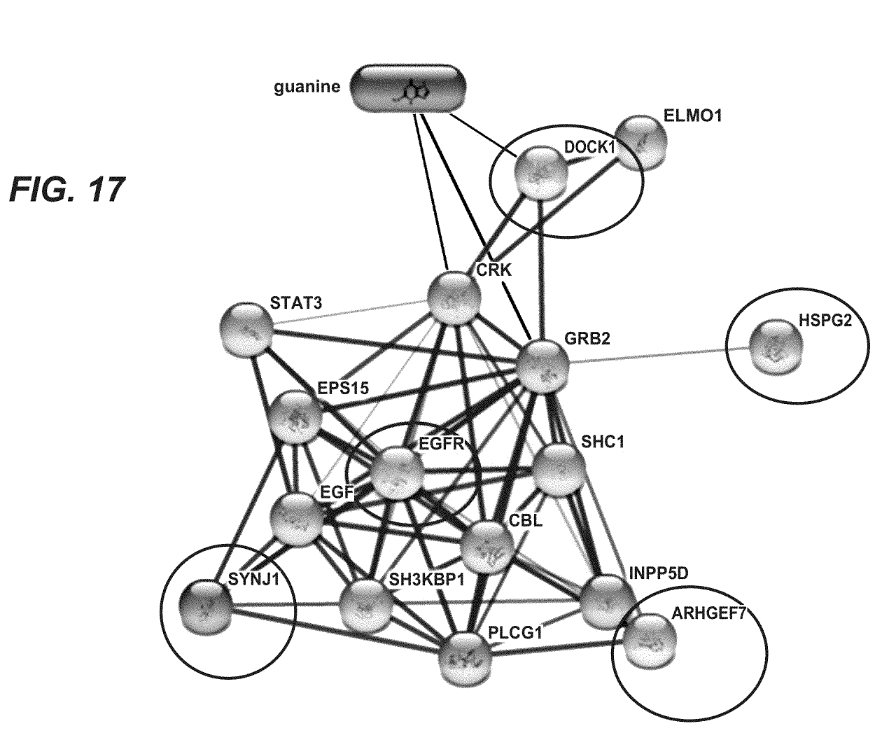

A total of 23 genes were implicated at the suggestive level (p<10⁻⁵). Of these, five interact closely with each other and with guanine (FIG. 17). The genes in question were ARHGEF7 (Rho Guanine Nucleotide Exchange Factor 7), DOCK1 (Dedicator of Cytokinesis 1), HSPG2 (Heparan Sulfate Proteoglycan 2), SYNJ1 (Synaptojanin 1), and EGFR (Epidermal Growth Factor). It is noted that guanine nucleoside analogues under investigation in the treatment of hepatitis B (Rivkin, 2007) and EGFR has been shown to interact with the hepatitis B virus (Menzo et al, 1993).

### Software

Custom-written perl scripts (Wall et al, 1996) were used to reformat the data, select markers for ancestry analysis and produce tables. PLINK version 1.07 (Purcell et al, 2007) was used to perform the genetic QC analyses, to merge study data with HapMap data, and for association analysis. EIGENSOFT 4.0 (Patterson et al, 2006; Price et al, 2006) was used for PCA. R version 2.15.2 (R Core Team, 2012) was used for the production of graphics.

### References

Devlin b, Roeder K (1999). Genomic control for association studies. Biometrics 55(4): 997-1004.
Dienstage J L (2008). Hepatitis B Virus Infection. N Engl J Med 359:1486-1500.
Dunteman G H (1989). Principal Components Analysis. SAGE university papers: Quantitative Applications in the Social Sciences. Series Editor: M S Lewis-Beck. Sage Publications Inc.
Ge D, Fellay J, Thompson A J, Simon J S, Shianna K V, Urban T J, Heinzen E L, Qiu P, Bertelsen A H, Muir A J, Sulkowski M, McHutchison J G, Goldstein D B (2009). Genetic variation in IL28B predicts hepatitis C treatment-induced viral clearance. Nature 161: 399-401.
Guo X, Zhang Y, Li J et al (2011). Strong influence of human leukocyte antigen (HLA)-DP gene variants on development of persistent chronic hepatitis B virus carries in the Han Chinese population. Hepatology 53: 422-8.
The International HapMap Consortium (2003). The International HapMap Project. Nature 426: 789-796.
The International HapMap Consortium (2005). A Haplotype Map of the Human Genome. Nature 437: 1299-1320.
The International HapMap Consortium (2007). A second generation human haplotype map of over 3.1 million SNPs. Nature 449:851-861.
Kamatani Y, Wattanapokayakit X, Ochi H et al (2009). a genome-wide association study identifies variants in the HLA-DP locus associated with chronic hepatitis B in Asians. Nat Genet 41: 591-595.
Menzo S, Clementi M, Alfani E, Bagnarelli P, Iacovacci S, Manzin A, Ddandri M, Natoli G, Levrero M, Carloni G (1993). Trans-activation of epidermal growth factor receptor gene by the hepatitis B virus X-gene product. Virology 196(2): 878-82.
Muller U, Steinhoff U, Reis LF, et al (1994). Functional role of type I and type II interferons in antiviral defence. Science 264: 1918-21.
Patterson N, Price A L, Reich D. Population structure and Eigenanalysis (2006). PLoS Genet. 2:e190.
Price A L, Patterson N J, Plenge R M, Weinblatt M E, Shadick N A, Reich D (2006). Principal components analysis corrects for stratification in genome-wide association studies. Nat Genet. 38:904.
Purcell S, Neale B, Todd-Brown K, Thomas L, Ferreira MAR, Bender D, Maller J, Sklar P, de Bakker P I W, Daly M J, Sham P C (2007). PLINK: a toolset for whole-genome association and population-based linkage analysis. Am J Hum Genet 81 (3):559-575.
R Core Team (2012). R: A language and environment for statistical computing. R Foundation for statistical computing, Vienna, Austria. ISBN 3-900051-07-0, URL http://www.R-project.org/)
Rivkin A (2007). Entecavir: a new nucleoside analogue for the treatment of chronic hepatitis B. Drugs Today (Barc) 43(4): 201-20.
Tanaka Y, Nishida N, Sugiyama M, Kurosaki M, Matsuura K, Sakamoto N, Nakagawa M, Korenaga M, Hino K, Hige S, Ito Y, Mita E, Tanaka E, Mochida S, Murawaki Y, Honda M, Sakai A, Hiasa Y, Nishiguchi S, Koike A, Sakaida I, Imamura M, Ito K, Yano K, Masaki N, Sugauchi F, Izumi N, Tokunaga K, Mizokami M (2009). Genome-wide association of IL28B with response to pegylated interferon-alpha and ribavirin therapy for chronic hepatitis C. Nat Genet 41(10): 1105-1109.
Wall L, Christiansen T, Schwartz R L (1996). Programming Perl (2^{nd} Edition). O'Reilly and Associates Inc (USA).
Whitlock M C (2005). Combining probability from independent tests: the weighted Z method is superior to Fisher's approach. J Evol Biol 18: 1368-1373.
WHO Fact Sheet No. 204; Revised August 2008.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method for treating hepatitis B virus (HBV) infection in a patient, the method comprising:
(a) detecting the presence of at least one A allele at rs7633796 in gene CADPS on chromosome 3 in a sample obtained from the patient, and
(b) administering an effective amount of an anti-HBV treatment comprising an interferon to the patient, wherein the patient has at least one A allele at rs7633796 in gene CADPS on chromosome 3, thereby treating HBV infection in the patient.

2. A method of identifying a patient who may benefit from treatment with an anti-HBV treatment comprising an interferon, the method comprising detecting the presence of a single nucleotide polymorphism in gene CADPS on chromosome 3 in a sample obtained from the patient, wherein the presence of at least one A allele at rs7633796 indicates that the patient may benefit from treatment with the anti-HBV treatment.

3. A method for predicting hepatitis B ‘e’ antigen (HBeAg) seroconversion and HBV DNA greater than 2000 IU/ml at greater than or equal to 24-week follow-up of treatment (responders vs. non-responders) of an HBe-positive patient infected with HBV to interferon treatment comprising:
(a) providing a sample from the patient,
(b) detecting the presence of a single nucleotide polymorphism in gene CADPS on chromosome 3, and
(c) determining that the patient has a high response rate to interferon treatment measured as HBeAg seroconversion and HBV DNA greater than 2000 IU/ml at greater than or equal to 24-week follow-up of treatment (responders vs. non-responders) if at least one A allele at rs7633796 is present in the patient.

4. The method of claim 1, wherein the interferon is selected from the group of peginterferon alfa-2a, peginterferon alfa-2b, interferon alfa-2a, and interferon alfa-2b.

5. The method of claim 4, wherein the interferon is a peginterferon alfa-2a conjugate having the formula: 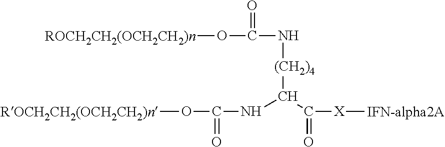
wherein R and R′ are methyl, X is NH, and n and n′ are individually or both either 420 or

520.

6. The method of claim 2, wherein the interferon is selected from the group of peginterferon alfa-2a, peginterferon alfa-2b, interferon alfa-2a, and interferon alfa-2b.

7. The method of claim 6, wherein the interferon is a peginterferon alfa-2a conjugate having the formula: 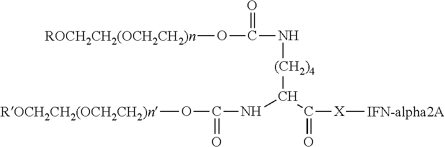
wherein R and R′ are methyl, X is NH, and n and n′ are individually or both either 420 or 520.

8. The method of claim 3, wherein the interferon is selected from the group of peginterferon alfa-2a, peginterferon alfa-2b, interferon alfa-2a, and interferon alfa-2b.

9. The method of claim 8, wherein the interferon is a peginterferon alfa-2a conjugate having the formula: 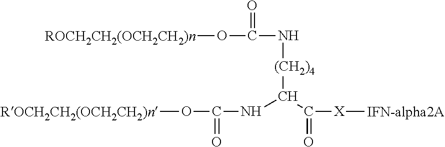
wherein R and R′ are methyl, X is NH, and n and n′ are individually or both either 420 or

520.
